(19) United States
(12) Patent Application Publication
(10) Pub. No.: US 2023/0330327 A1
(43) Pub. Date: Oct. 19, 2023

(54) PATCH INJECTION PUMP

(71) Applicant: INCUBE LABS, LLC, San Jose, CA (US)

(72) Inventor: Mir A. IMRAN, San Jose, CA (US)

(73) Assignee: INCUBE LABS, LLC, San Jose, CA (US)

(21) Appl. No.: 18/008,107

(22) PCT Filed: May 27, 2021
(86) PCT No.: PCT/US21/34613

§ 371 Date: Dec. 2, 2022

Related U.S. Application Data
(60) Provisional application No. 63/034,741, filed on Jun. 4, 2020.
(60) Provisional application No. 63/163,314, filed on Mar. 19, 2021.

(51) Int. Cl. A61M 5/142; A61M 5/145

(52) U.S. Cl. CPC A61M 5/14248; A61M 5/14526; A61M 2005/14252; A61M 2005/14272

(57) ABSTRACT

A patch pump includes a needle assembly, a reservoir, a preparation, and an inflatable component. The needle assembly includes a cannula, and a plunger slidably disposed in the cannula. The plunger has a pointed tip and is structured to move within the cannula upon application of a force on the plunger such that the pointed tip of the plunger extends from the cannula to form a channel in a tissue of a subject. The reservoir is fluidically coupled to the needle assembly, and the preparation is contained in the reservoir. The inflatable component is structured to, upon inflation, press against the reservoir to force the preparation from the reservoir and into a flowpath between the reservoir and the cannula.

### CROSS-REFERENCE TO RELATED APPLICATIONS

This application claims benefit of priority to Provisional U.S. patent application No. 63/034,741, filed Jun. 4, 2020, titled PATCH INJECTION PUMP; and to Provisional U.S. patent application No. 63/163,314, filed Mar. 19, 2021, titled PATCH INJECTION PUMP; both of the aforementioned priority applications being hereby incorporated by reference in their respective entireties.

### BACKGROUND

Injections generally are performed by first cleaning a site to be injected, filling a needle with liquid from a bottle, manually poking the needle through flesh, forcing the liquid into a subcutaneous area, removing the needle, and disposing of the needle. The process is time-consuming, requires contact between the subject and the caregiver giving the injection, and requires a store of medication bottles and needles. The process further requires experience in making injections. It would be beneficial to have an improved device for making injections, which device can be used even by inexperienced persons, where physical contact between a subject and a caregiver is not needed.

### SUMMARY

Embodiments of the present disclosure include a patch which, when triggered, provides a device for conveniently administering an automated injection of a medication.

Further details of these and other embodiments and aspects are described more fully below, with reference to the attached drawing figures.

### BRIEF DESCRIPTION OF THE DRAWINGS

FIG. 1A illustrates an embodiment of a patch pump shown from an interior top view with an upper portion of a housing omitted.

FIG. 1B illustrates an embodiment of the patch pump of FIG. 1A in a cross-sectional view along line A-A′ in FIG. 1A.

FIG. 1C illustrates an embodiment of the patch pump of FIG. 1A in a cross-sectional view along line B-B′ in FIG. 1A.

FIG. 2A illustrates an embodiment of an interior top view of the patch pump of FIG. 1A after the patch pump is triggered to administer an injection of medication, with the upper portion of the housing omitted.

FIG. 2B illustrates an embodiment of the patch pump of FIG. 2A in a cross-sectional view along line C-C′ in FIG. 2A.

FIG. 2C illustrates an embodiment of the patch pump of FIG. 2A in a cross-sectional view along line D-D′ in FIG. 2A.

FIG. 2D illustrates an embodiment of an extensor hinge of an inflatable component prior to inflation of the inflatable component.

FIG. 2E illustrates an embodiment of the extensor hinge of FIG. 2C after inflation of the inflatable component.

FIG. 3A illustrates an embodiment of an interior top view of the patch pump of FIG. 1A during administration of an injection of medication, with the upper portion of the housing omitted.

FIG. 3B illustrates an embodiment of the patch pump of FIG. 3A in a cross-sectional view along line E-E′ in FIG. 3A.

FIG. 3C illustrates an embodiment of the patch pump of FIG. 3A in a cross-sectional view along line F-F′ in FIG. 3A.

FIG. 4A, FIG. 4B, and FIG. 4C illustrate an embodiment of a progression of a needle assembly of a patch pump in an initial state, a state during deployment, and a state during delivery of a medication, respectively.

FIG. 4D and FIG. 4E illustrate an embodiment of the needle assembly of FIG. 4A.

FIG. 4F and FIG. 4G illustrate an embodiment of the needle assembly of FIG. 4B.

FIG. 4H and FIG. 4I illustrate an embodiment of the needle assembly of FIG. 4C.

FIG. 5A, FIG. 5B, and FIG. 5C illustrate an embodiment of a spring assembly in an initial state, a state during deployment, and a state during delivery of a medication, respectively.

FIG. 6A, FIG. 6B, and FIG. 6C illustrate an embodiment of a spring assembly in an initial state, a state during deployment, and a state during delivery of a medication, respectively.

FIG. 7A, FIG. 7B, and FIG. 7C illustrate an embodiment of spring assembly in an initial state, a state during deployment, and a state during delivery of a medication, respectively.

FIG. 8A illustrates an embodiment of a separation mechanism.

FIG. 8B illustrates an embodiment of a separation mechanism.

FIG. 8C and FIG. 8D illustrate embodiments of a closure.

FIG. 9 illustrates an embodiment of a patch pump from an interior top view with an upper portion of a housing omitted.

FIG. 10A, FIG. 10B, FIG. 10C, FIG. 10D, and FIG. 10E illustrate an embodiment of a needle assembly in various stages during deployment.

FIG. 11 illustrates relative dimensioning of an embodiment of the needle assembly of FIG. 10A-FIG. 10E in the various stages during the deployment.

FIG. 12 is a representative diagram illustrating an embodiment of a patch pump.

FIG. 13 is a representative diagram illustrating an embodiment of a patch pump.

FIG. 14A and FIG. 14B illustrate an embodiment of a dual-spring dual-axis needle assembly in perspective view, where FIG. 14B is a line drawing of the illustration in FIG. 14A.

FIG. 15A and FIG. 15B illustrate an embodiment of a cannula assembly in an assembled view and an exploded view, respectively.

FIG. 15C and FIG. 15D 15B illustrate an embodiment of a plunger assembly in an assembled view and an exploded view, respectively.

FIG. 15E illustrates an embodiment of a rail assembly.

FIG. 16A, FIG. 16B, and FIG. 16C illustrate an embodiment of a first phase of operation of the needle assembly of FIG. 14A and FIG. 14B.

FIG. 16D and FIG. 16E illustrate a comparison of an initial state (FIG. 16D) of the needle assembly of FIG. 14A and FIG. 14B versus a state of the needle assembly at or near an end of the first phase of operation (FIG. 16E).

FIG. 17A, FIG. 17B, and FIG. 17C illustrate an embodiment of a second phase of operation of the needle assembly of FIG. 14A and FIG. 14B.

FIG. 18A and FIG. 18B illustrate a third phase of operation of the needle assembly of FIG. 14A and FIG. 14B.

FIG. 19 illustrates a final state of the needle assembly of FIG. 14A and FIG. 14B after completion of the third phase of operation.

FIG. 20A, FIG. 20B, FIG. 20C, and FIG. 20D illustrate an embodiment of using a pinch valve to block flow of medication from a reservoir until a cannula is in a configuration suitable for providing treatment.

FIG. 21A illustrates an example of a design for a plunger.

FIG. 21B illustrates an example of a design for a plunger.

FIG. 22A, FIG. 22B, FIG. 22C, and FIG. 22D illustrate embodiments of a mechanism that can be used to activate a patch pump, such as to initiate balloon inflation and/or to initiate spring motion.

FIG. 23A and FIG. 23B illustrate exterior perspective and side views, respectively, of embodiments of a patch pump.

FIG. 23C illustrates an embodiment of the patch pump of FIG. 23B during an intermediary phase of operation.

FIG. 23D illustrates an embodiment of the patch pump of FIG. 23B at a final state, in a configuration suitable for providing treatment.

FIG. 24A, FIG. 24B, and FIG. 24C illustrate an embodiment of a dual-spring single-axis needle assembly in perspective view, where FIG. 24A illustrates the needle assembly in an initial state, FIG. 24B illustrates the needle assembly in an intermediary state of operation, and FIG. 24C illustrates the needle assembly in a final state.

FIG. 25A and FIG. 25B illustrate in cross-section embodiments of the needle assembly of FIG. 24A in side view (FIG. 25A) and top view (FIG. 25B).

FIG. 26A, FIG. 26B, and FIG. 26C illustrate an embodiment of a first phase of operation of the needle assembly of FIG. 24A.

FIG. 27A and FIG. 27B illustrate embodiments of the patch pump of FIG. 24A at a final state, in a configuration suitable for providing treatment.

### DETAILED DESCRIPTION

Described herein is a patch pump which may be placed against, or temporarily adhered to, skin of a subject to deliver a preparation to the subject. The patch pump can be placed on or against the skin by a caregiver or by the subject. The delivery mechanism is automated, and either the subject or the caregiver may initiate delivery of the preparation. Accordingly, the patch pump may be used by the subject without the subject coming near or into contact with a caregiver.

The patch pump is useful for many purposes, such as providing a vaccine, medication, or preparation to a subject. The patch pump is additionally useful in that it can be used in a variety of situations, such as when it is dangerous for caregivers to be near the subject (e.g., during a pandemic, when the subject has a contagious disease, or when the subject is immunosuppressed), or such as to provide preparations to remote geographies where there are no caregivers available, or such as to provide a supply of a preparation (e.g., insulin, adalimumab, or epinephrine) to a subject in a manner not requiring the subject to carry and use a needle and/or a bottle of the preparation, or such as to provide a convenient device for use in a doctor's office or nurse's station. Moreover, because a single-use patch pump is self-contained to deliver a dose or doses of a preparation, the subject does not see a needle, and therefore stress on the subject related to getting an injection may be reduced, and perhaps eliminated. This is especially important when treating children.

The patch pump can deliver multiple different preparations concurrently or sequentially, from different reservoirs within or fluidically coupled to the patch pump. In this manner, for example, multiple vaccinations and/or multiple medications may be given concurrently or sequentially without using multiple patch pumps.

The patch pump includes a needle assembly with a retractable pointed tip to create a short channel into and/or through subcutaneous tissue of the subject's body, and the patch pump includes a cannula to be positioned in the channel created by the pointed tip. A reservoir is fluidically coupled to the cannula to deliver a preparation from the reservoir through the cannula and into the body.

In an embodiment, an inflatable component is inflated upon the occurrence of a triggering event, and the inflation squeezes the reservoir to deliver the preparation through the cannula. The triggering event can be an action by a subject or a caregiver, such as pulling or pushing a tab, moving a switch, or sending a command from another device (e.g., smartphone, smartwatch, or dedicated controller) to electronics in the patch pump.

An embodiment of a patch pump may have any suitable shape, such as oval, circular, square, rectangular, or other polygonal cross-sectional shape, or such as an irregular shape (in vertical and/or horizontal cross-section). By way of example of irregular shapes, a housing may be in the shape of an animal or cartoon character or flower. Another example of an irregular shape is a housing having indents for ease of grasping the housing.

The patch pump has small dimensions. In an embodiment, the patch pump has a height of about 15.0 mm (e.g., about 0.59 inch), and an oval perimeter that is about 20.0 mm (e.g., about 0.79 inch) on a long axis and about 10.0 mm (e.g., about 0.39 inch) on a short axis. In an embodiment, the patch pump has a height of about 13.0 mm (e.g., about 0.51 inch) and a circular radius of about 38.0 mm (e.g., about 1.50 inches). Dimensions of the patch pump may be different for different embodiments, and are in part determined by an amount of a preparation to be delivered and a volume of a reservoir or reservoirs to be filled with preparation. In an embodiment, a volume of an internal reservoir is in a range between about 0.1 ml to about 10 ml (about 0.10 cc to about 10.0 cc). In an embodiment, a volume of an internal reservoir is in a range between about 0.25 ml to 1.0 ml (about 0.25 cc to about 1.0 cc).

The discussion is now directed to several embodiments of the patch pump illustrated by the figures. Other embodiments will be apparent to one skilled in the art after reviewing the figures and descriptions thereof, and all such embodiments are within the scope of the present disclosure.

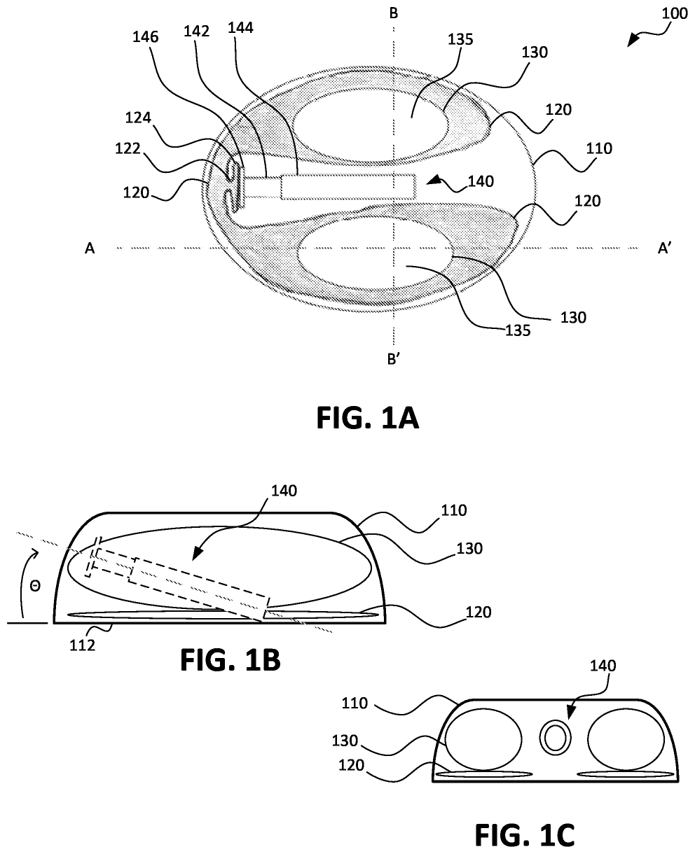

FIG. 1A illustrates an interior top view of an embodiment of a patch pump 100 in an initial state, with a top portion of a housing 110 omitted for ease of illustration. FIG. 1B illustrates an embodiment of the patch pump of FIG. 1A in a cross-sectional view along line A-A′ in FIG. 1A. FIG. 1C illustrates an embodiment of the patch pump of FIG. 1A in a cross-sectional view along line B-B′ in FIG. 1A.

Referring to FIG. 1A-FIG. 1C, the patch pump 100 encloses an inflatable component 120, at least one reservoir 130 (two are shown), and a needle assembly 140.

The inflatable component 120 is uninflated initially, as is indicated in FIG. 1B and FIG. 1C. The inflatable component 120 includes an extensor hinge 122 and a driver 124. Although illustrated in a horseshoe-like configuration with the extensor hinge 122 and the driver 124 in approximately a middle of the configuration, the inflatable component 120 may have a different shape, and the extensor hinge 122 and the driver 124 may be located off-center, or towards an end of the inflatable component 120.

The reservoir 130 contains a preparation, referred to for purposes of discussion as a medication 135, and the reservoir 130 is fluidically coupled to the needle assembly 140. In an embodiment including two or more reservoirs 130, all reservoirs 130 may contain a same or similar medication 135, or at least one of the multiple reservoirs 130 may contain a medication 135 different than a medication 135 contained in another of the reservoirs 130.

The needle assembly 140 includes a plunger 142, a cannula 144 in which the plunger 142 is slidably disposed, and a header 146 affixed to, or integral to, the plunger 142. The needle assembly 140 is positioned at an angle ‘0’ from a lower surface 112 of the housing 110 (FIG. 1B).

The driver 124 of the inflatable component 120 abuts the header 146 of the needle assembly 140 in the initial state illustrated in FIG. 1A.

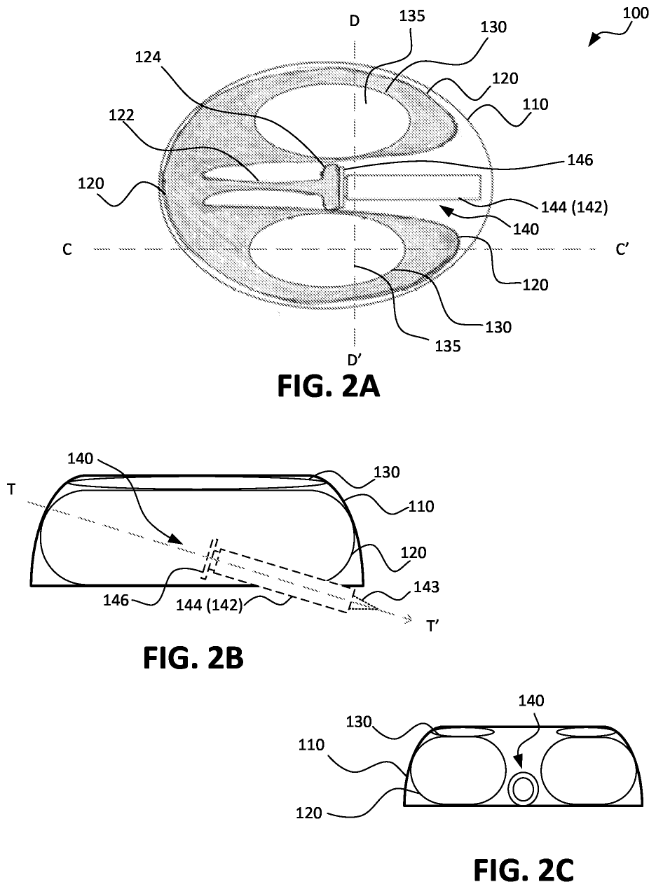

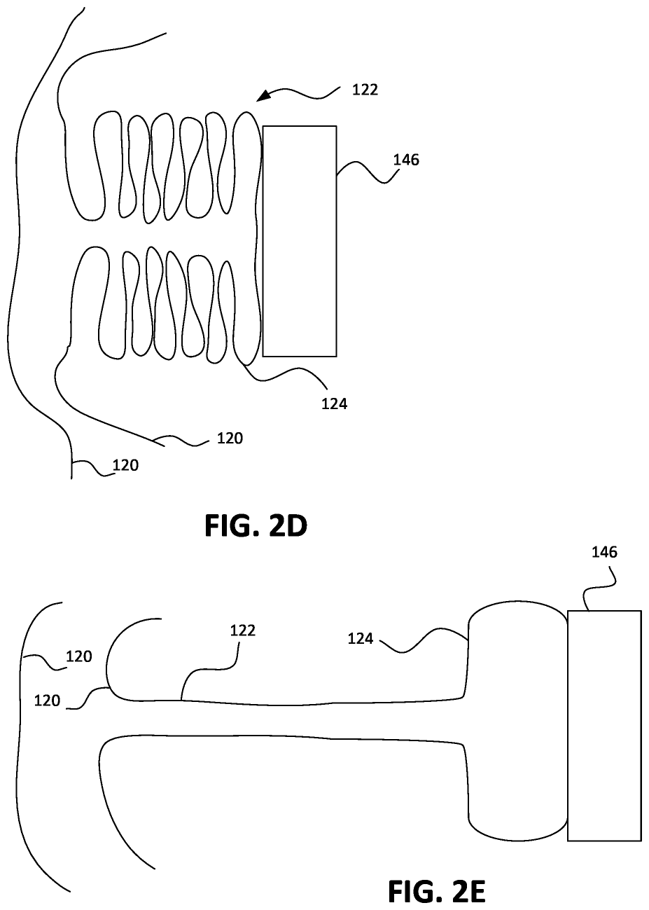

FIG. 2A illustrates an interior top view of an embodiment of the patch pump 100 of FIG. 1A (again omitting the top portion of the housing 110 for ease of illustration) after the patch pump 100 is triggered to administer an injection of the medication 135. FIG. 2B illustrates an embodiment of the patch pump 100 of FIG. 2A in a cross-sectional view along line C-C′ in FIG. 2A. FIG. 2C illustrates an embodiment of the patch pump of FIG. 2A in a cross-sectional view along line D-D′ in FIG. 2A.

Referring to FIG. 2A-FIG. 2C, in response to a triggering event, the inflatable component 120 has inflated. As illustrated in FIG. 2A, the extensor hinge 122 has inflated and extends in a relatively narrow column to the driver 124, which has also inflated.

In an embodiment, the extensor hinge 122 as inflated has approximately a columnar shape such that the extensor hinge 122 has an approximately elliptical or circular shape in cross-section. In an embodiment, the extensor hinge 122 has an approximately polygonal shape in cross-section. The extensor hinge 122 may have a consistent cross-sectional shape and/or dimension along its length as inflated, or may have a varying cross-sectional shape and/or dimension along its length as inflated.

In an embodiment, the driver 124 as inflated has an approximately elliptical or circular shape in cross-section. In an embodiment, the driver 124 has an approximately polygonal shape in cross-section. The driver 124 may have a consistent cross-sectional shape and/or dimension along its length as inflated, or may have a varying cross-sectional shape and/or dimension along its length as inflated.

The inflation of the extensor hinge 122 and the driver 124 exerts force against the header 146 of the needle assembly 140, which in turn exerts force against the plunger 142 such that the plunger 142 slides within the cannula 144, and the entirety of the needle assembly 140 is pushed along a trajectory as indicated by the line T-T′ of FIG. 2B. Although the trajectory is indicated as a straight line in FIG. 2B, the trajectory could alternatively be arcuate, such as by adjusting an angle at which the driver 124 of the inflatable component 120 exerts force against the plunger 142, or by adjusting a shape of the extensor hinge 122 to inflate in an arcuate manner.

The plunger 142 includes a sharp pointed tip 143 affixed to, or formed integrally with, the plunger 142. In an initial state (e.g., FIGS. 1A-1C), the tip 143 may be fully within the cannula 144 or may extend beyond an end of the cannula 144. In an embodiment, the tip 143 extends about 0.8 mm (e.g., about 0.03 inch) beyond the end of the cannula 144 in the initial state. In an embodiment, the tip 143 is flush with or recessed from the end of the cannula 144 in the initial state. In an embodiment, the tip 143 extends from the end of the cannula 144 by less than about 0.25 mm (e.g., less than about 0.01 inch), or between about 0.25 mm to about 2.5 mm (e.g., between about 0.01 inch to about 0.10 inch).

As the needle assembly 140 is pushed along the trajectory T-T′ or other trajectory by force of the driver 124 against the header 146, the plunger 142 moves through the cannula 144 and the tip 143 is exposed (or further exposed) from the cannula 144 (FIG. 2B). The tip 143 pierces tissue adjacent to the housing 110 as the needle assembly 140 is pushed along the trajectory, and the tip 143 creates a channel into or through subcutaneous tissue. The cannula 144 enters the tissue channel.

FIG. 2D illustrates an embodiment of the extensor hinge 122 of the inflatable component 120 prior to inflation of the inflatable component 120 (e.g., as in FIGS. 1A-1C). The extensor hinge 122 is accordioned or folded into a pre-deployed state. The driver 124 of the inflatable component 120 is positioned against the header 146 of the needle assembly 140.

FIG. 2E illustrates an embodiment of the extensor hinge 122 of FIG. 2D after inflation of the inflatable component 120 (e.g., as in FIGS. 2A-2C). The extensor hinge 122 during inflation pushes the header 146 along the trajectory until the extensor hinge 122 is fully or near fully extended. During inflation, the extensor hinge 122 provides an increasing force. After inflation, the extensor hinge 122 is compliant (e.g., bendable) due to having a relatively small cross-sectional dimension.

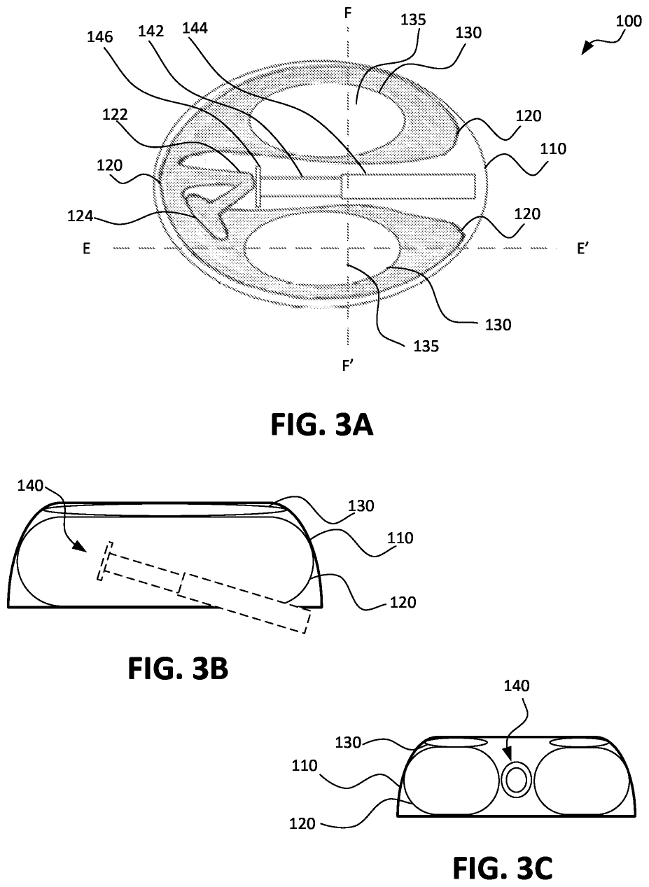

FIG. 3A illustrates an interior top view of an embodiment of the patch pump 100 of FIG. 1A (again omitting the top portion of the housing 110 for ease of illustration) during administration of an injection of the medication 135. FIG. 3B illustrates an embodiment of the patch pump of FIG. 3A in a cross-sectional view along line E-E′ in FIG. 3A. FIG. 3C illustrates an embodiment of the patch pump of FIG. 3A in a cross-sectional view along line F-F′ in FIG. 3A.

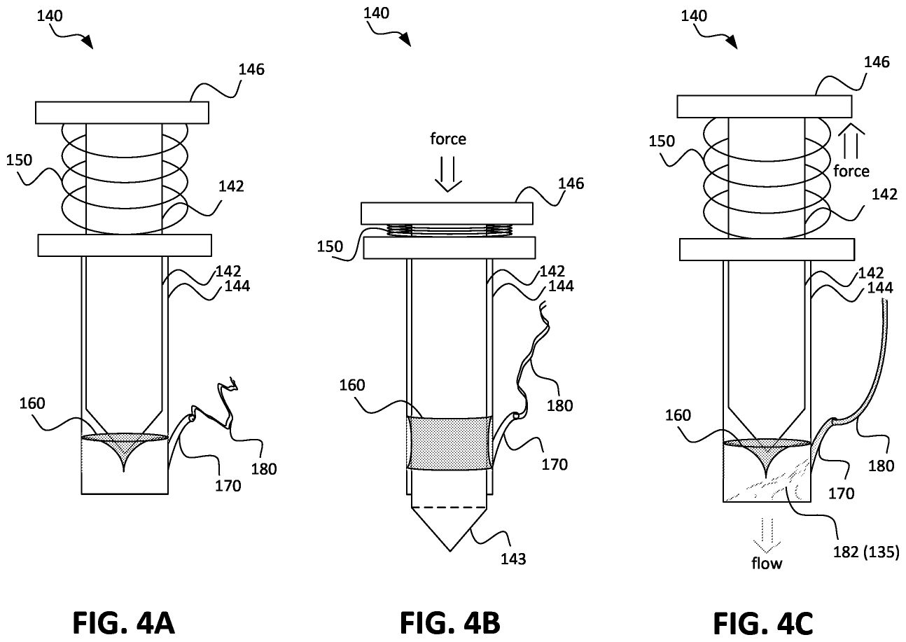

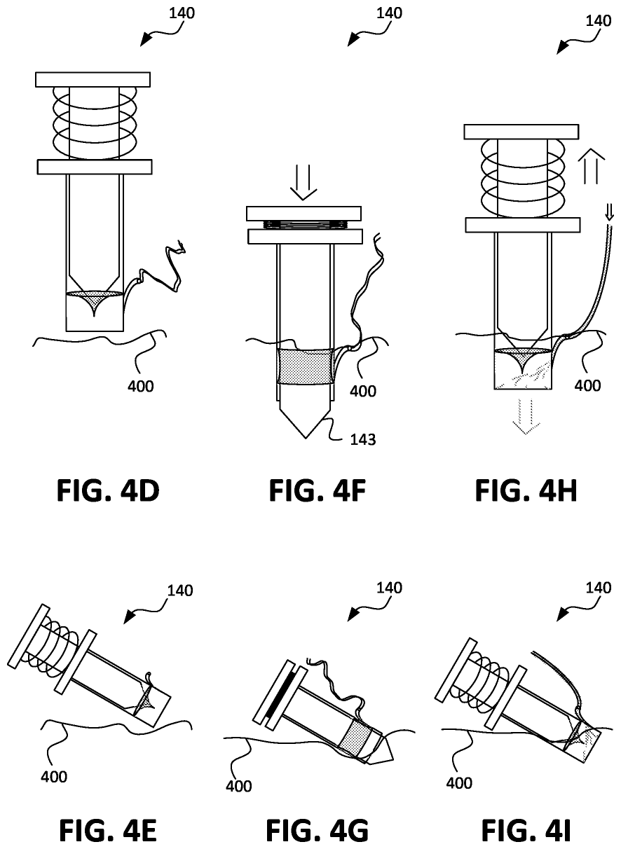

Referring to FIG. 3A-FIG. 3C, after the cannula 144 enters the channel formed by the tip 143, the plunger 142 is forced back into the cannula 144 such that the tip 143 is no longer exposed from the cannula 144. Examples of techniques for forcing the plunger 142 back into the cannula 144 are illustrated and described with respect to FIGS. 4A-7C. Because the extensor hinge 122 is compliant, the plunger 142 can push the driver 124 and the extensor hinge 122 out of the way as it travels back through the cannula 144 (FIG. 3A). The cannula 144 remains in the channel in a configuration ready to deliver the medication 135.

Meanwhile, as the inflatable component 120 inflates, the inflatable component 120 also exerts force on the reservoir 130 to squeeze the medication 135 out of the reservoir 130 and through the cannula 144.

FIG. 4A-FIG. 4C illustrate an embodiment of the needle assembly 140 of the patch pump 100, as it may appear in an initial state (FIG. 4A), a state during deployment (FIG. 4B), and a state during retraction of the plunger 142 and delivery of the medication 135 (FIG. 4C). In an embodiment, the initial state may approximately correspond to FIGS. 1A-1C, the state during deployment may approximately correspond to FIGS. 2A-2C, and the state during retraction of the plunger 142 and the delivery of the medication may approximately correspond to FIGS. 3A-3C.

With reference to FIG. 4A, the needle assembly 140 includes a spring 150, a valve 160, and a port 170. One end of a tube 180 is fluidically coupled to the port 170. The other end of the tube 180 is coupled (not shown) to the reservoir 130. In an embodiment, multiple tubes 180 are fluidically coupled to the port 170, and all of the tubes 180 are fluidically coupled to a single reservoir 130, or at least one of the tubes 180 is fluidically coupled to a reservoir 130 that is different from a reservoir 130 to which another of the tubes 180 is fluidically coupled. The needle assembly 140 is illustrated in an initial state in which the spring 150 is in a relaxed state, the valve 160 is closed, and the port 170 and the tube 180 are empty.

With reference to FIG. 4B, expansion of the inflatable component 120 (not shown) causes the driver 124 (not shown) to exert force against the header 146. The header 146 in turn exerts force against the plunger 142, which then slides through the cannula 144 through the valve 160 to expose the tip 143 from the cannula 144 and form a channel through tissue. The cannula 144 enters the channel. The force exerted by the header 146 against the plunger 142 and the resulting movement of the plunger 142 compress the spring 150.

With reference to FIG. 4C, as the plunger 142 reaches an end of travel along the trajectory, the spring force of the spring 150 pushes the header 146 and thus the plunger 142 in approximately an opposite direction from the previous path of motion of the plunger 142, to pull the tip 143 back into the cannula 144 so that the tip 143 is no longer exposed from the cannula 144. By this technique, the tip 143 is contained within the housing 110 except for a short period of time (e.g., in terms of milliseconds or seconds) when the tip 143 extends sufficiently beyond the cannula 144 to create a channel through tissue. Accordingly, the tip 143 does not pose a danger to a person handling the patch pump 100. The plunger 142 and its tip 143 are pulled back by the spring 150 sufficiently far for the valve 160 to close. The medication 135 is allowed to flow through the tube 180, into and through the port 170, into and through the cannula 144 (flow 182 in FIG. 4C), and into tissue. After a time as determined by a structure of the patch pump 100, a quantity of the medication 135 has been delivered, and the patch pump 100 can be removed, by pulling the patch pump 100 away from the skin of the subject, thus pulling the cannula 144 out of the channel in the tissue.

FIG. 4D and FIG. 4E further illustrate the needle assembly 140 of FIG. 4A. FIG. 4D is similar to FIG. 4A, illustrating the needle assembly 140 adjacent to tissue 400 from a view representative of an interior top view of the patch pump 100. FIG. 4E is a side view of the needle assembly 140 of FIG. 4D illustrating how the needle assembly 140 might sit in relation to the tissue 400 in an initial state.

FIG. 4F and FIG. 4G further illustrate the needle assembly 140 of FIG. 4B. FIG. 4F is similar to FIG. 4B, illustrating the tip 143 of the plunger 142 forming a channel in the tissue 400 from a view representative of an interior top view of the patch pump 100. FIG. 4G is a side view of the needle assembly 140 of FIG. 4F illustrating how the tip 143 might advance in relation to the tissue 400 during deployment.

FIG. 4H and FIG. 4I further illustrate the needle assembly 140 of FIG. 4C. FIG. 4H is similar to FIG. 4C, illustrating the cannula 144 in the channel formed in the tissue 400 and the plunger 142 retracted into the cannula 144 past the valve 160, from a view representative of an interior top view of the patch pump 100. FIG. 4I is a side view of the needle assembly 140 of FIG. 4H illustrating how the needle assembly 140 might appear during delivery of the medication 135 to tissue.

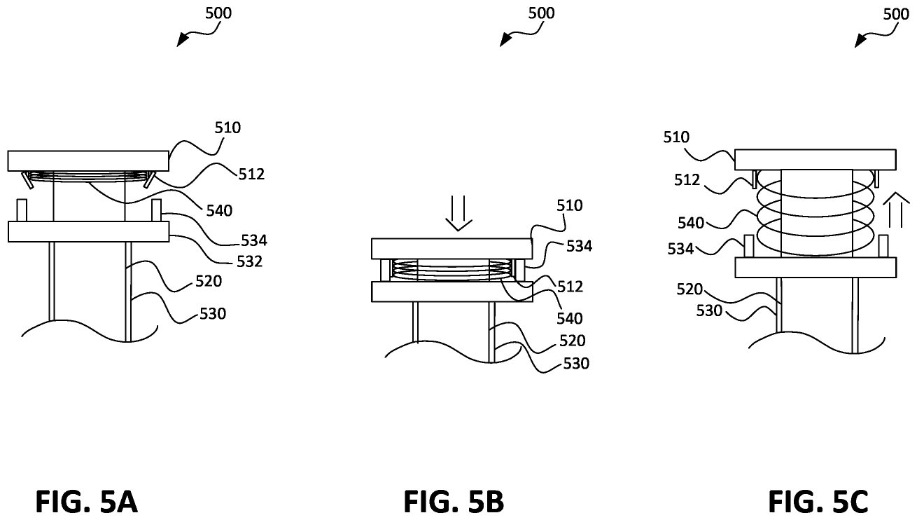

FIGS. 5A-FIG. 5C illustrate an embodiment of a spring mechanism of a needle assembly 500 (e.g., similar to the needle assembly 140) in an initial state (FIG. 5A), a state during deployment (FIG. 5B), and a state during retraction of (FIG. 5C). The needle assembly 500 includes a header 510, a plunger 520, a cannula 530, and a spring 540. The header 510 includes one or more hinged clamps 512 (two are shown). The cannula 530 includes a platform 532 and one or more guideposts 534 attached to, or integrally formed with, the platform 532. A number of the guideposts 534 is equal to or greater than a number of the hinged clamps 512, or the guidepost 534 is a single component with a continuous perimeter around the platform 532.

With reference to FIG. 5A, the spring 540 is held in an initial compressed position against the header 510 by the hinged clamps 512.

With reference to FIG. 5B, expansion of the inflatable component 120 (not shown) causes the driver 124 (not shown) to exert force against the header 510. The header 510 in turn exerts force against the plunger 520, which then slides through the cannula 530 to form a channel through tissue and the cannula 530 enters the channel (as described above). The force exerted by the header 510 against the plunger 520 and the resulting movement of the plunger 520 compress the spring 540. As force is applied to the header 510 and the header 510 moves and exerts force against the plunger 520, the hinged clamps 512 engage the guideposts 534 in a manner such as to release the spring 540 from the hinged clamps 512.

With reference to FIG. 5C, once released, the spring 540 exerts force against the header 510, causing the plunger 520 to retract (e.g., slide within the cannula 530 until a pointed tip (not shown) of the plunger 520 is no longer exposed from the cannula 530).

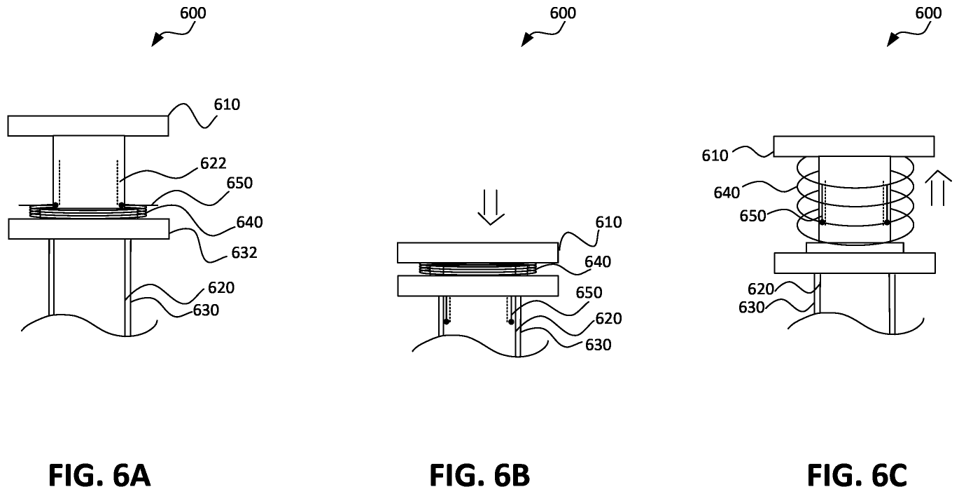

FIG. 6A-FIG. 6C illustrate an embodiment of a spring mechanism of a needle assembly 600 (e.g., similar to the needle assembly 140) in an initial state (FIG. 6A), a state during deployment (FIG. 6B), and a state during retraction (FIG. 6C). The needle assembly 600 includes a header 610, a plunger 620, a cannula 630, and a spring 640. The plunger 620 defines one or more grooves 622 (two are shown). The needle assembly 600 further includes one or more lock pins 650. For each of the lock pins 650, one end of the lock pin 650 is disposed in a groove 622 and the lock pin 650 rotates around that end. The cannula 630 includes a platform 632.

With reference to FIG. 6A, the spring 640 is held in an initial compressed position against the platform 632 by the lock pins 650, which are in a position approximately parallel to the platform 632.

With reference to FIG. 6B, expansion of the inflatable component 120 (not shown) causes the driver 124 (not shown) to exert force against the header 610. The header 610 in turn exerts force against the plunger 620, which then slides through the cannula 630 to form a channel through tissue and the cannula 630 enters the channel (as described above). As the plunger 620 moves within the cannula 630, the lock pins 650 are forced to rotate around the ends disposed within the grooves 622 such that the lock pins 650 are each in a position approximately perpendicular to the platform 632.

With reference to FIG. 6C, when the lock pins 650 rotate into the grooves 622 and no longer oppose motion of the spring 640, the spring 640 exerts force against the header 610, causing the plunger 620 to retract (e.g., slide within the cannula 630 until a pointed tip (not shown) of the plunger 620 is no longer exposed from the cannula 630).

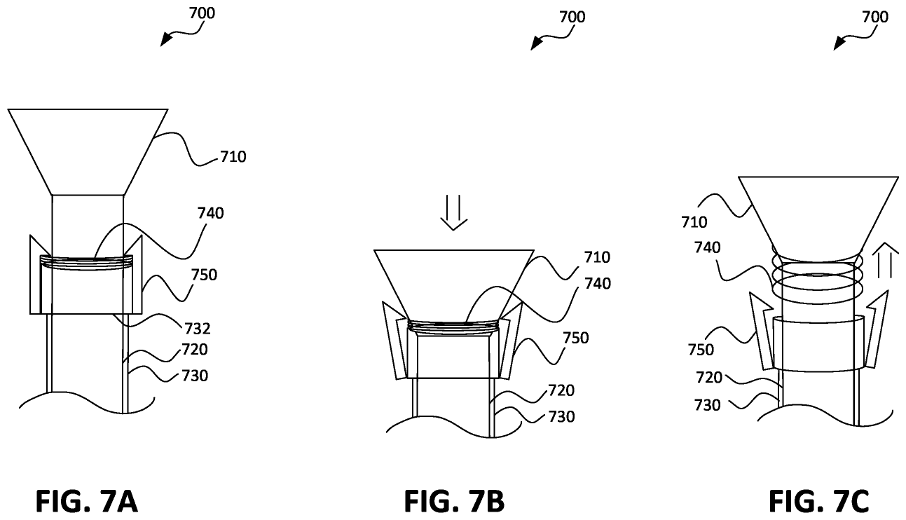

FIG. 7A-FIG. 7C illustrate an embodiment of a spring mechanism of a needle assembly 700 (e.g., similar to the needle assembly 140) in an initial state (FIG. 7A), a state during deployment (FIG. 7B), and a state during retraction (FIG. 7C). The needle assembly 700 includes a header 710, a plunger 720, a cannula 730, and a spring 740. The cannula 730 includes a platform 732 and one or more lock arms 750 attached to, or integrally formed with, the platform 732 in a hinging manner.

With reference to FIG. 7A, the spring 740 is held in an initial compressed position against the platform 732 by the lock arms 750.

With reference to FIG. 7B, expansion of the inflatable component 120 (not shown) causes the driver 124 (not shown) to exert force against the header 710. The header 710 in turn exerts force against the plunger 720, which then slides through the cannula 730 to form a channel through tissue and the cannula 730 enters the channel (as described above). As the plunger 720 moves within the cannula 730, the header 710 engages the lock arms 750. The header 710 has an angled shape such that the header 710 spreads the lock arms 750 apart.

With reference to FIG. 7C, when the lock arms spread apart and no longer oppose motion of the spring 740, the spring 740 exerts force against the header 710, causing the plunger 720 to retract (e.g., slide within the cannula 730 until a pointed tip (not shown) of the plunger 720 is no longer exposed from the cannula 730).

In an embodiment, to inflate an inflatable component (e.g., the inflatable component 120), a patch pump (e.g., the patch pump 100) includes two or more reactants that, when combined, form a gas to accomplish the inflation. For example, citric acid and sodium bicarbonate react to form a carbon dioxide gas. The reactants are kept separate until it is desired to initiate an injection (e.g., of the medication 135). In an embodiment, a patch pump includes a separation mechanism coupled to a tab; the separation mechanism keeps two or more reactants separate. When the tab is pulled (e.g., removed), the reactants mix and form a gas to inflate the inflatable component. Pulling the tab is thus a triggering event for delivering the preparation to the subject in this embodiment.

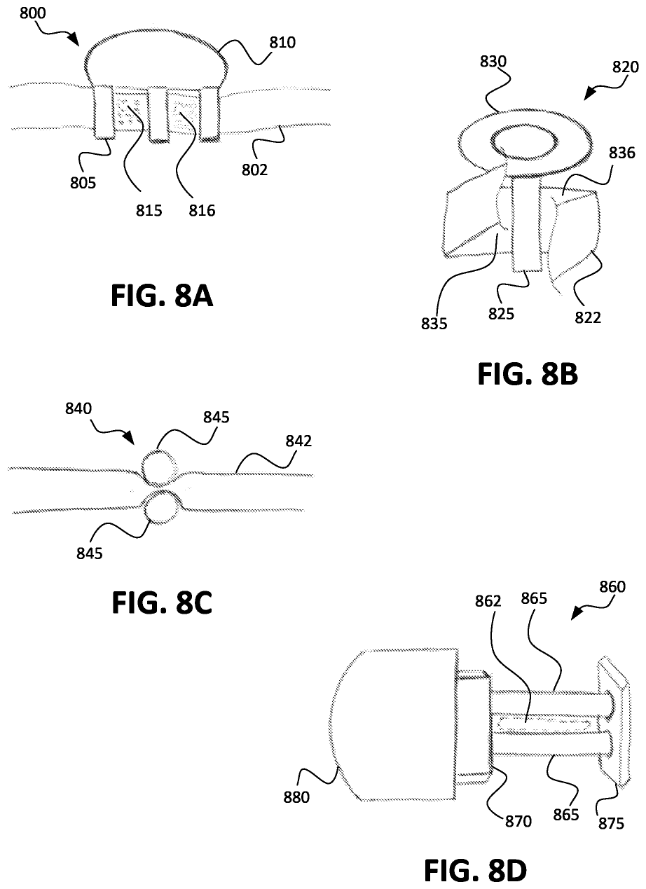

FIG. 8A illustrates an embodiment of a separation mechanism 800 including three closures 805 closing off an inflatable component 802, a tab 810 mechanically coupled to the closures 805, and a first reactant 815 and a second reactant 816 disposed in the inflatable component 802. The closures 805 keep the first reactant 815 and the second reactant 816 contained in defined areas in the inflatable component 802 and separate from each other. When the tab 810 is pulled, the closures 805 release and allow the first reactant 815 and the second reactant 816 to mix and form a gas to inflate the inflatable component 802.

FIG. 8B illustrates an embodiment of a separation mechanism 820 including one closure 825 closing off an inflatable component 822, a tab 830 mechanically coupled to the closure 825, a first reactant disposed in a portion 835 of the inflatable component 822, and a second reactant disposed in a portion 836 of the inflatable component 822. The closure 825 keeps the portion 835 and the portion 836 separated (and thus keeps the first reactant and the second reactant separated from each other). The inflatable component 822 can be folded to form the portion 835 between a first fold and the closure 825 and to form the portion 836 between a second fold and the closure 825. When the tab 830 is pulled, the closure 825 releases and allows the first reactant and the second reactant to mix and form a gas to inflate the inflatable component 822.

FIG. 8C illustrates an embodiment of a closure 840 (e.g., the closure 805 or the closure 825) in which two members 845 squeeze an inflatable component 842 closed.

FIG. 8D illustrates an embodiment of a closure 860 (e.g., the closures 805, the closure 825, or the closure 840) in which two members 865 squeeze an inflatable component 862 closed. The members 865 are mechanically coupled to a block 870 and are engaged with a plate 875. A tab 880 is attached to the block 870. When the tab 880 is pulled, the members 865 are disengaged from the plate 875 so that the inflatable component 862 is released (e.g., reactants are allowed to mix to form a gas to inflate the inflatable component 862).

A reservoir of the patch pump (e.g., the reservoir 130 of the patch pump 100) is fluidically coupled to a port (e.g., the port 170, through the tube 180) of a needle assembly (e.g., the needle assembly 140) to provide a flowpath for a preparation (e.g., the medication 135) to the port and thereby to a subject. To avoid having the preparation or biological matter enter the flowpath in a reverse direction through the port and the tube, the needle assembly may include a port valve in the port and/or in the tube. The port valve is a one-way valve. In an embodiment, the port valve includes a duck-bill valve. Such a port valve is in addition to a valve (e.g., the cannula valve 160) which keeps a pointed tip (e.g., the tip 143) from being exposed from the needle assembly and also keeps biological matter or preparation from flowing into the patch pump rather than out of the needle assembly into tissue.

A patch pump (e.g., the patch pump 100) may be affixed to an adhesive material which may be adhered to skin of a subject (e.g., to an arm, leg, back, stomach, or buttock of the subject) to administer an injection. In an embodiment, the adhesive material defines an opening through which a needle assembly (e.g., the needle assembly 140) may protrude when deployed to deliver preparation (e.g., the medication 135). In an embodiment, the adhesive material defines an opening which is covered by a seal to maintain a clean or aseptic environment within the patch pump until injection. A pointed tip (e.g., the tip 143) can first pierce the seal and then pierce tissue of the subject. In an embodiment, the adhesive material is a foam with an adhesive across all or a portion of the side of the foam to be positioned against skin of the subject, and an adhesive on an opposite side of the foam sized to be disposed under and affix to the patch pump housing.

In an embodiment, a patch pump (e.g., the patch pump 100) is gamma sterilized prior to being filled and sealed, the preparation is filled into a reservoir (e.g., the reservoir 130) in an aseptic environment, and the patch pump is sealed before removal from the aseptic environment.

In an embodiment, a patch pump (e.g., the patch pump 100) is sterilized and sealed and disposed in packaging, all within a sterile or aseptic environment. In an embodiment, multiple patch pumps are disposed in the packaging. In an embodiment, a single patch pump is disposed in the packaging. In an embodiment, a sterilizing wipe is included with the packaging so that a user may sterilize the packaging prior to opening the packaging; for example, a caregiver may sterilize the packaging, provide the patch pump to a subject, and the subject may open the packaging and position and trigger the patch pump to deliver the medication contained in the patch pump, to minimize exposure of the subject to germs, viruses, toxins, chemicals, or other substance on the packaging.

In an embodiment, a patch pump (e.g., the patch pump 100) includes electronics to detect, store, and/or report to another device information such as proximity of the patch pump to skin, removal of a tab (e.g., the tab 810, 830, or 880), inflation of an inflatable component (e.g., the inflatable component 120, 802, 822, 842, or 862), deployment of a needle assembly (e.g., the needle assembly 140, 500, 600, or 700), delivery of a quantity of preparation (e.g., the medication 135), an expected amount of preparation remaining in one or more reservoirs (e.g., the reservoir 130), a notification that a refill of a reservoir is needed, time, temperature, oxygen content, pulse rate, or other parameter related to the patch pump or to a subject against which the patch pump is pressed or to an environment of the patch pump. Appropriate sensors are incorporated into the patch pump to monitor the desired parameters.

In an embodiment including a port valve to keep biological matter or preparation from flowing into a flowpath, the port valve is a controllable valve, and the electronics control the port valve to open and close, such as periodically with a fixed or variable duty cycle, or pulsed once or in a pulse train at certain preset times after a tab is pulled, or upon occurrence of an event (e.g., blood oxygen or insulin levels crossing a threshold).

In an embodiment, alternative to or additional to a reservoir (e.g., the reservoir 130), a patch pump (e.g., the patch pump 100) may include a coupling with a one-way valve such that a preparation may be injected into the coupling and thus into a flowpath and to a port (e.g., the port 170) to deliver the preparation to tissue of a subject. The coupling can also be used to attach an external supply of preparation such as contained in a bag (e.g., a gravity-drip bag or a pressurized bag).

In an embodiment, a pointed tip of a needle assembly (e.g., the tip 143 of the plunger 142 of the needle assembly 140) has a diameter similar to a 28-gauge needle. In an embodiment, a maximum dimension (e.g., diameter) of a plunger (e.g., the plunger 142) is less than a diameter of a 28-gauge needle.

In an embodiment, a pointed tip of a needle assembly (e.g., the tip 143 of the plunger 142 of the needle assembly 140) advances about 5 mm to about 6 mm (e.g., between about 0.19 inch and 0.24 inch) or more into tissue when deployed, and a cannula 144 also advances about 5 mm to about 6 mm or more into the tissue.

In an embodiment, a dimension (e.g., a diameter, length, or width) of a housing of a patch pump (e.g., the housing 110 of the patch pump 100) is less than a diameter of quarter, and preferably less than a diameter of a nickel. In an embodiment, the housing has a circular perimeter with diameter less than about 25 mm (e.g., about 0.98 inch). In an embodiment, the housing has an elliptical perimeter with a short axis less than about 21 mm (e.g., about 0.83 inch).

In an embodiment, a patch pump (e.g., the patch pump 100) is affixed to an adhesive material, and a dimension of the adhesive material is less than twice a dimension of the patch pump, and preferably less than 1.1 times a dimension of the patch pump. For example, the adhesive material may be affixed to a lower surface of a housing of the patch pump (e.g., the lower surface 112 of the housing 110) and may not be visible, or may be somewhat visible, around a perimeter of the patch pump.

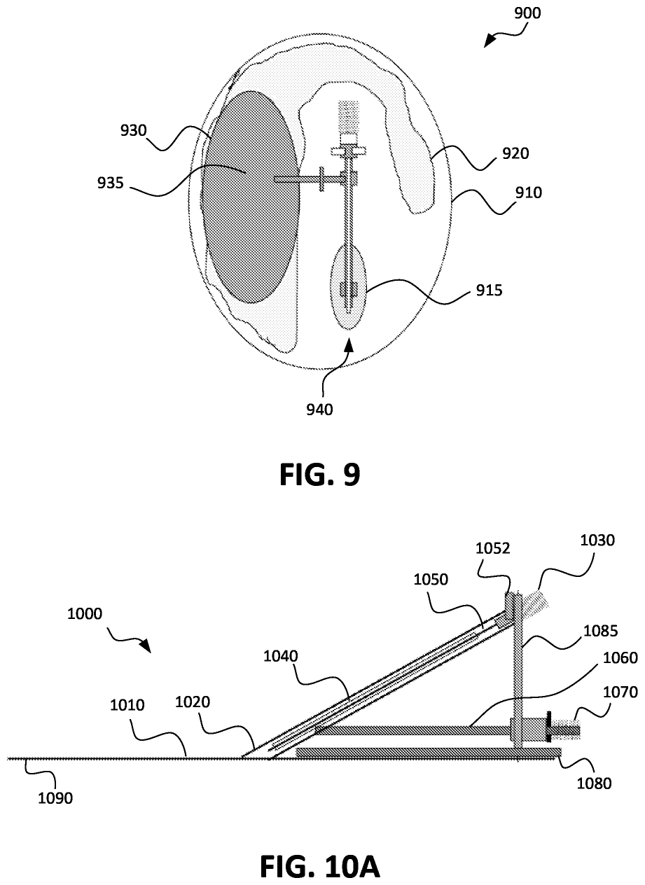

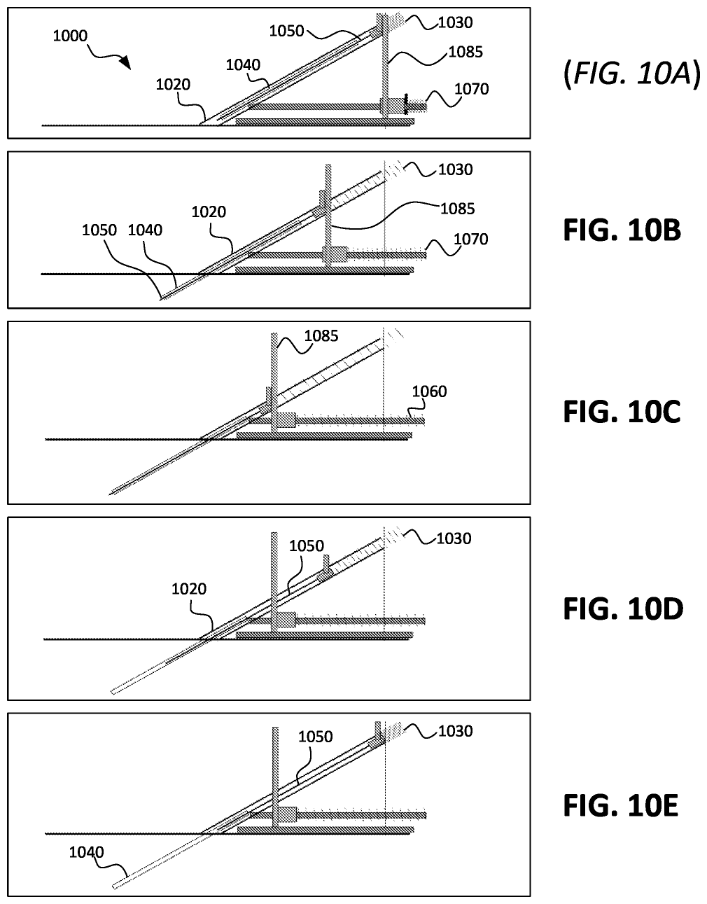

FIG. 9 and FIG. 10A-FIG. 10E together illustrate an embodiment (with variations) of a patch pump. In this embodiment, the needle assembly is dual-spring and dual-axis, with a compression spring that is extended in its relaxed state and can be biased into a compressed state and an extension spring that is not extended in its relaxed state and can be biased into an extended state.

Initially, the compression spring is biased into a state of full or partial compression, and the extension spring is unbiased or substantially unbiased (e.g., in its relaxed state). The extension spring is coupled to a plunger of a needle assembly disposed in the patch pump. The compression spring is coupled to the extension spring. Upon the occurrence of an event, the compression spring is released from bias and extends towards its unbiased state, which biases the extension spring into an extended state and causes the plunger and a cannula to move. Movement of the compression spring deploys the needle assembly into tissue of a body (e.g., a pointed tip of the plunger advances into the tissue and creates a channel in the tissue, and the cannula enters the channel to deliver a preparation). After deployment, the compression spring is uncoupled from the extension spring, the compression spring subsequently remains in its unbiased state, and the extension spring returns to its unbiased state (not extended). As the extension spring returns to its unbiased state, the extension spring pulls the plunger back into the cannula.

Referring to FIG. 9, a patch pump 900 is illustrated from an interior top view, omitting a top portion of a housing 910 of the patch pump 900 for ease of illustration. The patch pump 900 includes a base 915, an inflatable component 920, a reservoir 930 containing a preparation, shown as a medication 935, and a needle assembly 940.

The base 915 is coupled to the housing 910 and holds a non-movable portion of the needle assembly 940 in a consistent position relative to the housing 910. The inflatable component 920 in many respects is similar to the inflatable component 120 of FIG. 1A-FIG. 3C, except that the inflatable component 920 does not have a hinge or a driver (e.g., the extensor hinge 122 and the driver 124 to contact and move the needle assembly 140). The reservoir 930 is similar to the reservoir 130 of FIG. 1A-FIG. 3C. Inflation of the inflatable component 920 squeezes the reservoir 930 to expel medication 935 from the reservoir 930 into a flowpath through the needle assembly 940, in a manner similar to inflatable component 120 squeezing reservoir 130 to expel medication 135 through the needle assembly 140 when inflatable component 120 is inflated, as described above.

Referring to FIG. 10A, a needle assembly 1000 (e.g., an embodiment of the needle assembly 940 in FIG. 9) includes a base 1010, a rail 1020, an extension spring 1030, a cannula 1040, a plunger 1050 having a header 1052, a bar 1060, a compression spring 1070, and a frame including a stand 1080 and a support 1085. The plunger 1050 is slidably disposed within the cannula 1040. The cannula 1040 is slidably disposed within or coupled to the rail 1020. The rail 1020 is coupled to the base 1010 and another portion of the patch pump (e.g., to the housing 910 or to the support 1085), to control or maintain an angle between the base 1010 and the rail 1020. One end of the extension spring 1030 is coupled to an end of the rail 1020, or is coupled to a housing (e.g., the housing 910), and an opposite end of the extension spring 1030 is coupled to the plunger 1050. The bar 1060 is coupled to the rail 1020. The compression spring 1070 is coupled to the support 1085. The support 1085 is slidably coupled to the stand 1080 and/or slidably positioned over the bar 1060, to allow the support 1085 to move along a path approximately parallel to the base 1010. The needle assembly 1000 is shown positioned adjacent skin 1090 of a subject.

In an embodiment, the rail 1020 includes at least one slot along its length. A protrusion affixed to or formed integrally with the header 1052 extends from a slot in the rail 1020 and is engaged by the support 1085, and a port affixed to or formed integrally with the cannula 1040 extends from a slot in the rail 1020 to provide a flowpath for the preparation.

Referring to FIG. 10A-FIG. 10E, an initial position (FIG. 10A, which is reproduced adjacent FIG. 10B-FIG. 10E for reference) and subsequent movement of the various components of the needle assembly 1000 (FIG. 10B-FIG. 10E) are shown, in an embodiment.

In FIG. 10A, the needle assembly 1000 is in an initial state prior to an occurrence of an event that initiates deployment. The event, when it occurs, will release the compression spring 1070 from its biased state.

In FIG. 10B, the compression spring 1070 has been released from its biased state. The compression spring 1070 pushes the support 1085, such that the plunger 1050 with the cannula 1040 are pushed along the rail 1020 and into and through a surface of the skin 1090 into tissue. Meanwhile, movement of the support 1085 pulls the extension spring 1030 into its biased state. The compression spring 1070 is sufficiently stronger than the extension spring 1030 to overcome the combined forces of: the tendency of the extension spring 1030 to return to its unbiased state; component friction of the needle assembly 1000; and resistance of the skin 1090.

In FIG. 10C, the support 1085 reaches an end of travel along the bar 1060.

In FIG. 10D, at the end of travel of the support 1085, the plunger 1050 is disengaged from the support 1085. In an embodiment including a slot in the rail 1020 through which a protrusion of the plunger 1050 extends, the slot extends along a straight line parallel to an axis of the rail 1020 until at or near the end of travel of the support 1085; the slot then deviates from the straight line, which causes the plunger 1050 to rotate due to the protrusion of the plunger 1050 following the deviated path of the slot; rotation of the plunger 1050 disengages the plunger 1050 from the support 1085. When the plunger 1050 is disengaged from the support 1085, the extension spring 1030 is allowed to return toward its unbiased state.

In FIG. 10E, the return of the extension spring 1030 to its unbiased state results in the plunger 1050 being pulled back into the patch pump. The cannula 1040 remains in the tissue to deliver the preparation.

Occurrence of an event that initiates deployment may be, for example, pulling or pushing of a tab, pushing on a lever or switch, or receiving a signal from an external device to electronics in the patch pump to trigger deployment. In an embodiment, a tab is pulled or pushed to inflate an inflatable component (e.g., as described with respect to FIG. 8A-FIG. 8D), and the inflation disengages a mechanism holding the compression spring 1070 in a biased state, thus initiating deployment.

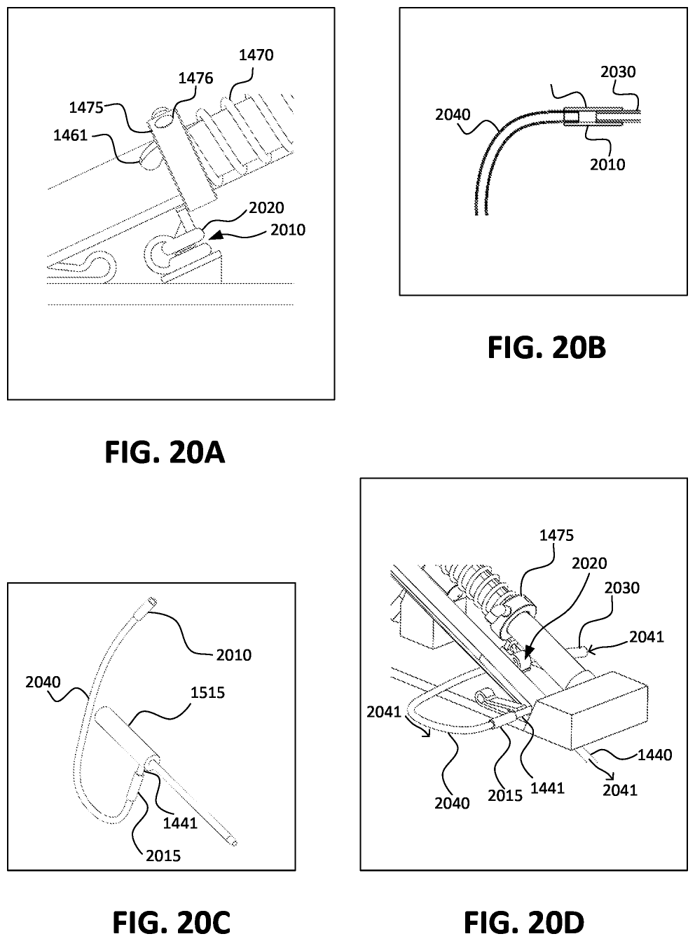

In an embodiment, a clamp is disposed to close off a flowpath coupled to the port of the cannula 1040; as the support 1085 moves towards (FIG. 10B) or is close to or reaches (FIG. 10C) its end of travel, the port comes into contact with and disengages the clamp, allowing a preparation to flow through the flowpath into the cannula 1040 and thus into the tissue (e.g., similar to the mechanisms described with respect to FIG. 20A-FIG. 20D).

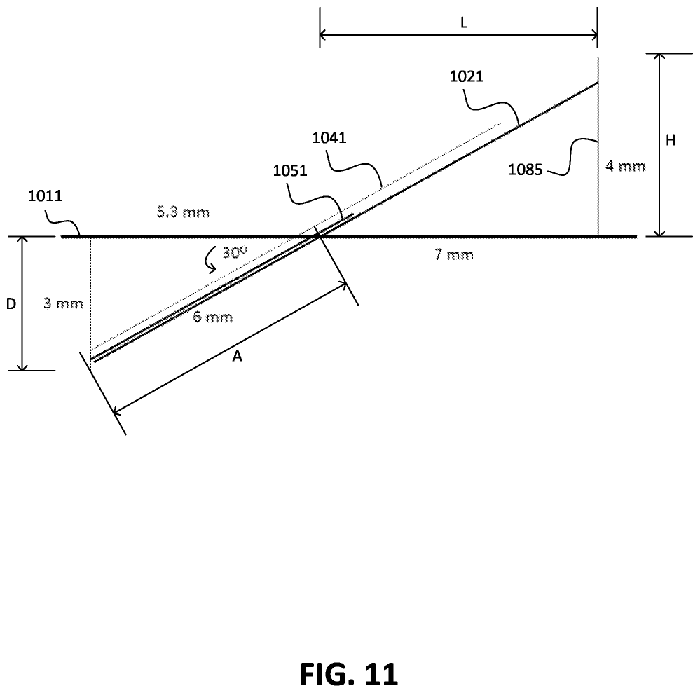

FIG. 11 indicates relative dimensioning of an embodiment of the needle assembly of FIG. 10A-FIG. 10E at an initial state and after deployment. In the embodiment illustrated, a length ‘L’ representing a length of the needle assembly 1000 in an initial state is about 7 mm (e.g., about 0.28 inch), a height ‘H’ representing a height of the needle assembly 1000 is about 4 mm (e.g., about 0.16 inch). A line 1021 is provided in FIG. 11 for reference and approximately aligns with a lower surface of the rail 1020 above the base 1010 (and is extended past the base 1010 for visual reference). A trajectory 1041 represents a trajectory of the cannula and a trajectory 1051 represents a trajectory of the plunger 1050. The needle assembly is structured for the cannula 1040 to be advanced along the trajectory 1041 to a distance ‘A’ of about 6 mm (e.g., about 0.24 inch) within the tissue after deployment if deployment occurs as designed, such as to a depth ‘D’ of about 3 mm (e.g., about 0.12 inch) for the angle illustrated. As illustrated, the trajectory 1051 and the trajectory 1041 in this embodiment make an approximately 30 degree angle with the lower surface 1011 of the base 1010. Other relative dimensioning is within the scope of the present disclosure. For example, the angle may be about 15 degrees to about 75 degrees, or a length or height of the needle assembly 1000 may be shorter or longer than indicated. In an embodiment, the angle is about 25 degrees.

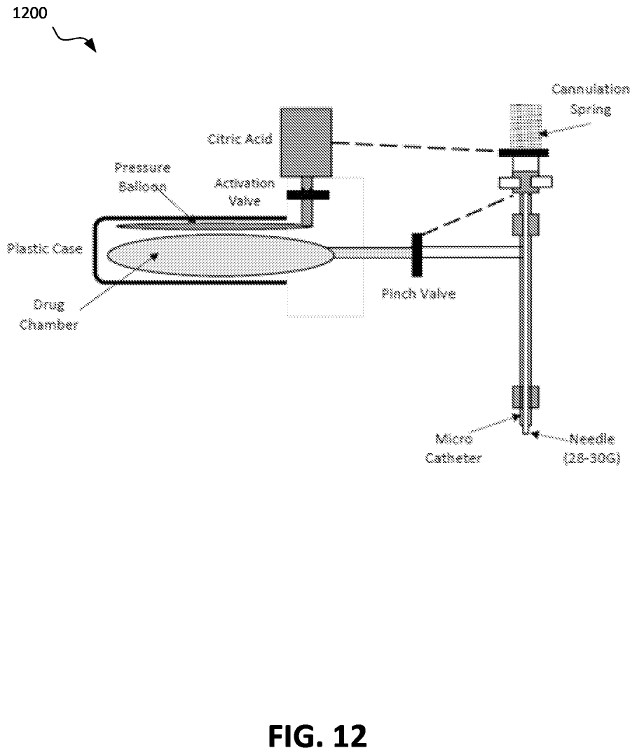

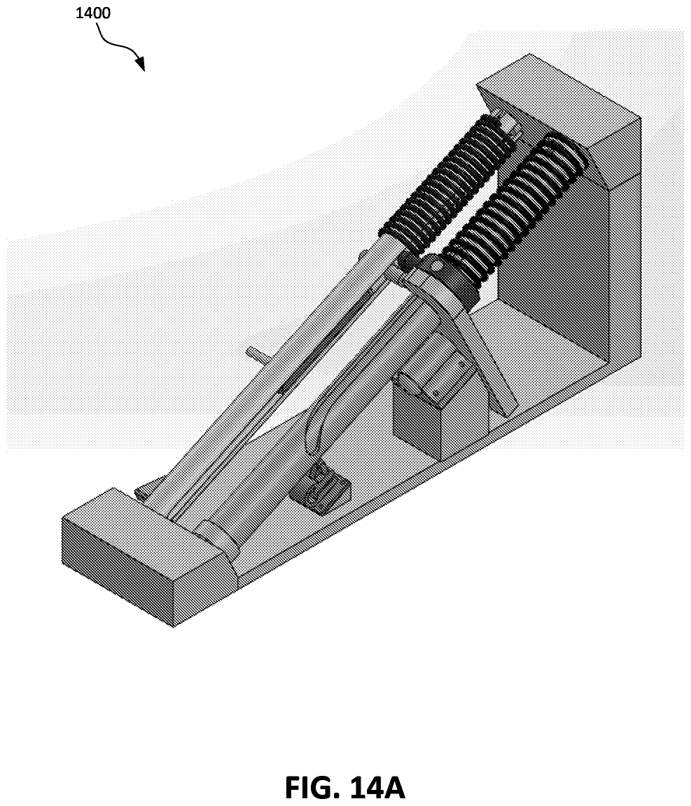

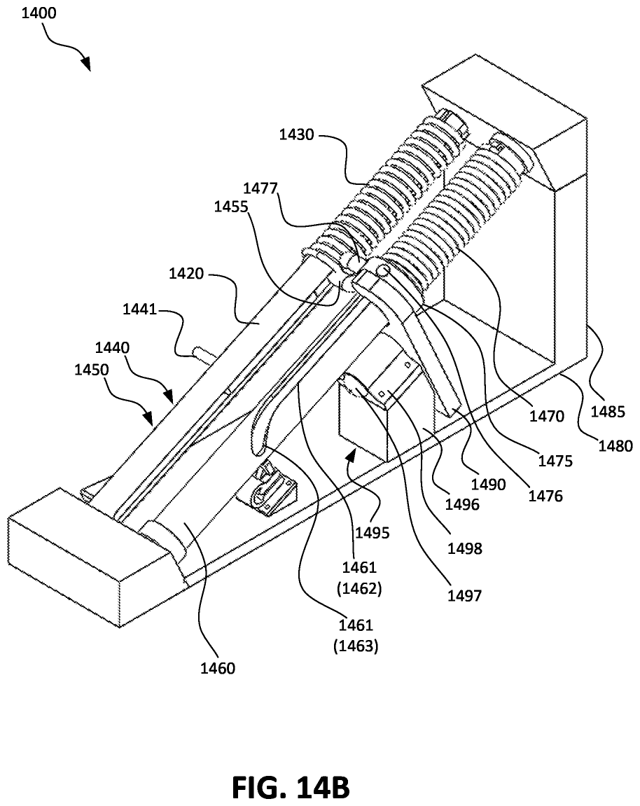

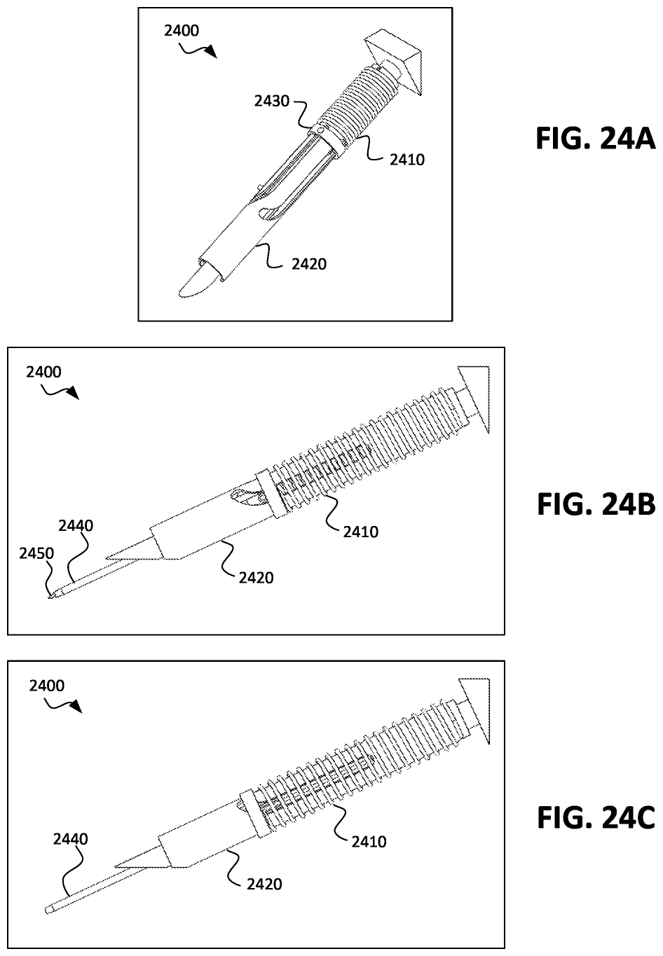

FIG. 12 is a descriptive block diagram illustrating an embodiment of a patch pump 1200 incorporating a needle assembly (e.g., the needle assembly 1000 of FIG. 10A-FIG. 10E, the needle assembly 1400 of FIG. 14B and related embodiments, or the needle assembly 2400 in FIG. 24A and related embodiments). In this embodiment, the terms “micro catheter”, “needle”, “drug chamber”, “plastic case”, and “pressure balloon” respectively refer to the terms cannula, plunger, reservoir, housing, and inflatable component as used herein.

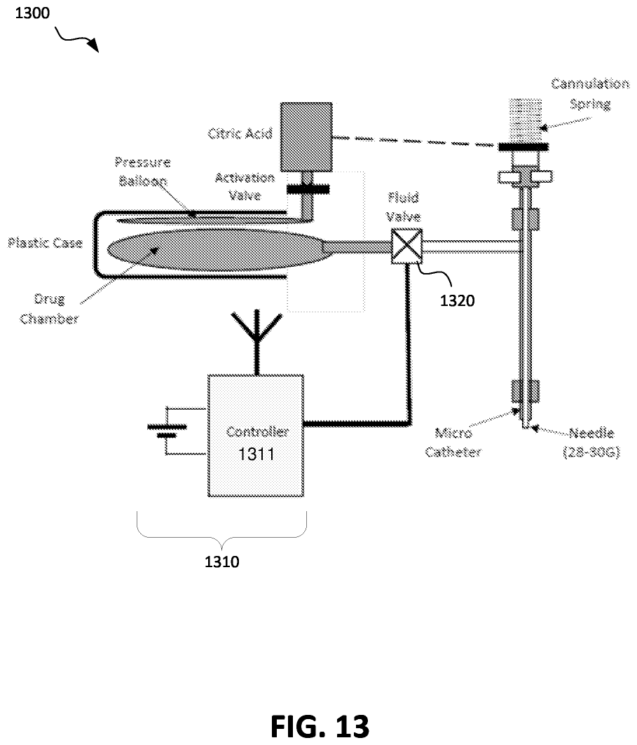

FIG. 13 is a descriptive block diagram illustrating an embodiment of a patch pump 1300 incorporating a needle assembly (e.g., the needle assembly 1000 of FIG. 10A-FIG. 10E, the needle assembly 1400 of FIG. 14B and related embodiments, or the needle assembly 2400 in FIG. 24A and related embodiments). This embodiment is similar in many respects to the patch pump 1200 in FIG. 12, and further includes electronics 1310 (including a controller 1311, power source and other circuitry) for controlling a valve 1320 (e.g., fluidic valve 1320) to deliver a preparation through the needle assembly according to a program stored in the electronics.

FIG. 14A-FIG. 20D together illustrate another embodiment (with variations) of a patch pump. In this embodiment, the needle assembly is dual-spring and dual-axis, with a compression spring that is extended in its relaxed state and can be biased into a compressed state and an extension spring that is not extended in its relaxed state and can be biased into an extended state. These embodiments are similar to needle assembly 1000 illustrated in FIG. 10A-FIG. 10E in many respects, with one difference being that the compression and extension springs in FIG. 14-FIG. 20D move along parallel trajectories rather than at an angle with respect to each other as is the case for needle assembly 1000.

Initially, the compression spring is biased into a state of full or partial compression, and the extension spring is unbiased or substantially unbiased (e.g., in its relaxed state). The extension spring is coupled to a plunger of a needle assembly disposed in the patch pump. The compression spring is coupled to the extension spring. Upon the occurrence of an event, the compression spring is released from bias and extends towards its unbiased state, which biases the extension spring into an extended state and causes the plunger and a cannula to move. Movement of the compression spring deploys the needle assembly into tissue of a body (e.g., a pointed tip of the plunger advances into the tissue and creates a channel in the tissue, and the cannula enters the channel to deliver a preparation). After deployment, the compression spring is uncoupled from the extension spring, the compression spring subsequently remains in its unbiased state, and the extension spring returns to its unbiased state (not extended). As the extension spring returns to its unbiased state, the extension spring pulls the plunger back into the cannula.

Referring to FIG. 14A, a needle assembly 1400 (e.g., a variation on the needle assembly 940 in FIG. 9) is shown in perspective view.

Referring to FIG. 14B, the needle assembly 1400 includes a rail 1420, an extension spring 1430, a cannula 1440, a plunger 1450, a bar 1460, a compression spring 1470, a bushing 1475, a frame including a stand 1480 and a support 1485, a retainer 1490, and a positioner 1495. The plunger 1450 is slidably disposed within the cannula 1440. The cannula 1440 is slidably disposed within the rail 1420. The rail 1420 is coupled to the frame (e.g., coupled to the stand 1480 and/or coupled to the support 1485). One end of the extension spring 1430 is coupled to an end of the rail 1420, or is coupled to the support 1485, and an opposite end of the extension spring 1430 is coupled to the plunger 1450. The bar 1460 is coupled to the frame (e.g., coupled to the stand 1480 and/or coupled to the support 1485). One end of the compression spring 1470 is coupled to an end of the bar 1460, or is coupled to the support 1485, and an opposite end of the compression spring 1470 is coupled to or is adjacent to the bushing 1475. The support 1485 is stationary with respect to the stand 1480.

In an embodiment, the stand 1480 and/or the support 1485 are coupled to a housing (not shown) of the patch pump. In an embodiment, the needle assembly is coupled directly to the housing and the frame is omitted, or the stand 1480 is omitted, or the support 1485 is omitted.

The stand 1480 and the housing, or the housing alone if the stand 1480 is omitted, each define an opening (not shown) through which the cannula 1440 and the plunger 1450 extend beyond an outer surface of the housing to pierce skin of a subject and thus deliver a preparation through the port 1441 and the cannula 1440.

A cannula assembly includes the cannula 1440 and a port 1441. The port 1441 is fluidically coupled to an internal or external reservoir (not shown).

A plunger assembly includes the plunger 1450 and a first interlock 1455.

The bar 1460 defines a slot 1461 having a substantially straight portion 1462 and a curved portion 1463.

A bushing assembly includes the bushing 1475, a slot post 1476, and a second interlock 1477. The slot post 1476 extends into the slot 1461 of the bar 1460. The slot post 1476 travels within and is guided by the slot 1461 as the bushing 1475 moves along the bar 1460.

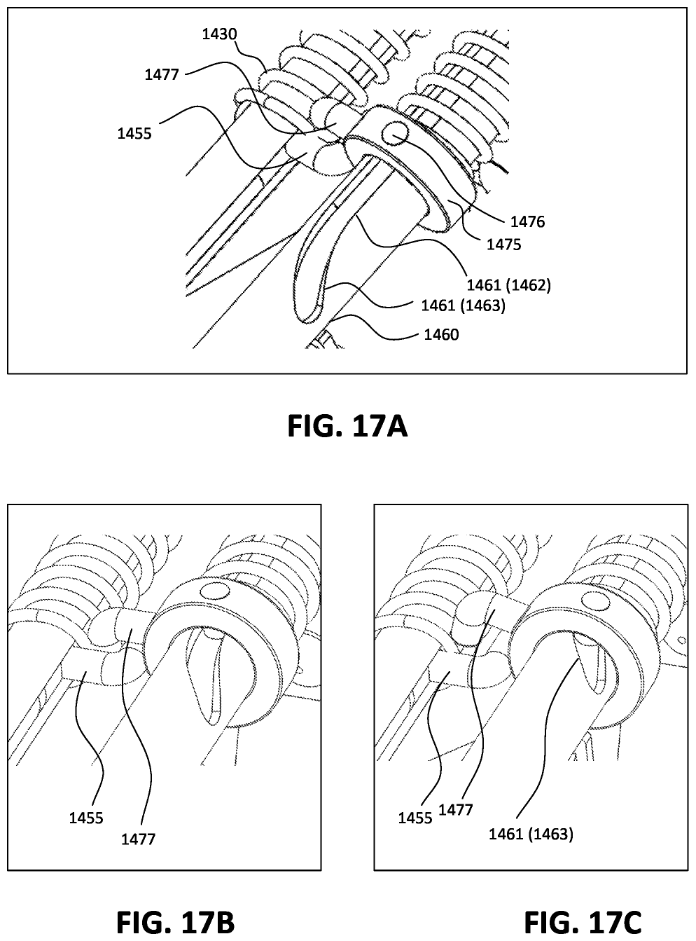

The retainer 1490 retains the bushing 1475 at an initial position, thus maintaining the compression spring 1470 in a biased (compressed) state. When the retainer 1490 is moved out of the way, such as by pulling or pushing a tab (not shown), the compression spring 1470 is allowed to decompress, pushing the bushing 1475 along the bar 1460 and thus moving the slot post 1476 along the slot 1461. Initially, and for a time after the compression spring 1470 begins pushing the bushing 1475 along the bar 1460, the first interlock 1455 and the second interlock 1477 are coupled together (e.g., as illustrated in FIG. 17A). In this manner, the decompression of the compression spring 1470 forces the extension spring 1430 into a biased (extended) state.

The positioner 1495 positions, and allows rotation of, the retainer 1490. The positioner 1495 includes an offset block 1496 to position the retainer 1490 as desired with respect to the bar 1460 and the bushing 1475, an axis pin 1497 to allow the retainer 1490 to rotate around an axis defined by a length of the axis pin 1497, and a clamp 1498 to firmly hold the axis pin 1497 to the offset block 1496. In an embodiment, the offset block 1496 can be variably positioned on the stand 1480, such as to reduce or increase a compression force against the compression spring 1470, and/or to reduce or increase a force needed to move the retainer 1490 to allow the compression spring 1470 to decompress.

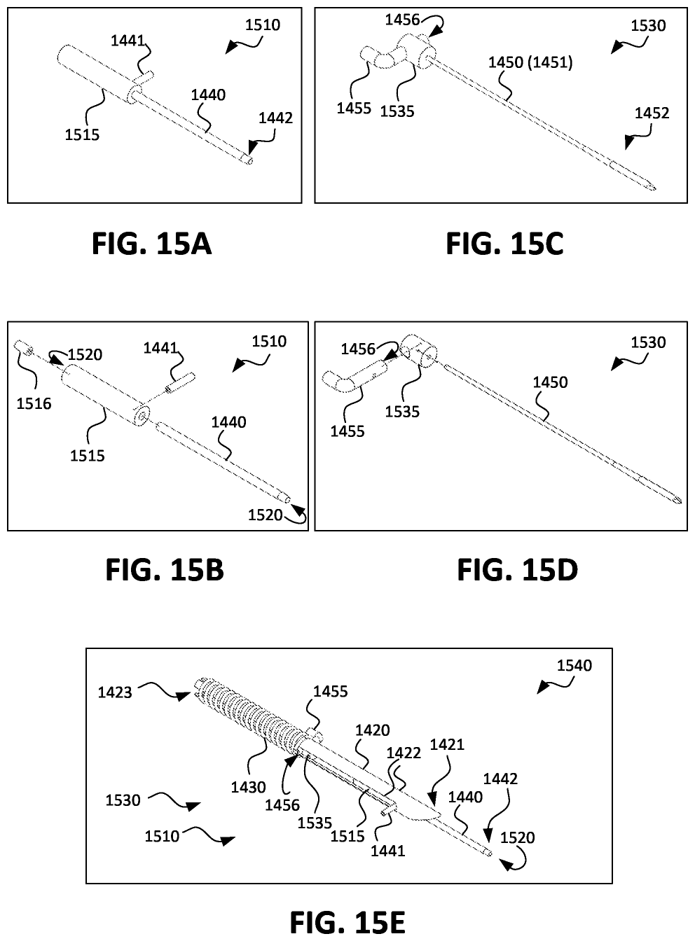

Referring to FIG. 15A and FIG. 15B, a cannula assembly 1510 is shown in assembled (FIG. 15A) and exploded (FIG. 15B) views. The cannula assembly 1510 includes, in an embodiment, the cannula 1440, the port 1441, and a cannula hub 1515. The cannula 1440 is fluidically coupled to the port 1441. The cannula assembly 1510 defines a duct 1520 that extends through the cannula 1440 and the cannula hub 1515. In an embodiment, an end 1442 of the cannula 1440 has a larger or smaller cross-sectional dimension (e.g., an outer or inner diameter) than a cross-sectional dimension (e.g., a respective outer or inner diameter) of a remainder of the cannula 1440. In an embodiment, the end 1442 has a smaller cross-sectional outer dimension that a cross-sectional outer dimension of the remainder of the cannula 1440 to assist in movement of the cannula into the tissue channel formed by the plunger 1450. A plug 1516 is disposed within the cannula hub 1515.

Referring to FIG. 15C and FIG. 15D, a plunger assembly 1530 is shown in assembled (FIG. 15C) and exploded (FIG. 15D) views. The plunger assembly 1530 includes, in an embodiment, the plunger 1450, the first interlock 1455, and a plunger hub 1535. The first interlock 1455 is disposed through the plunger hub 1535 to expose from the plunger hub 1535 an opening 1456 of the first interlock 1455. In an embodiment, a length of the first interlock 1455 is exposed from the plunger hub 1535 as illustrated in FIG. 15B; in another embodiment, a surface of the first interlock 1455 defining the opening 1456 is aligned with or is recessed from a surface of the plunger hub 1535 such that the first interlock 1455 does not extend beyond the plunger hub 1535. In an embodiment, the plunger 1450 includes a body 1451 and a tip 1452, where the tip 1452 has a different cross-sectional dimension (e.g., an outer diameter) than a cross-sectional dimension (e.g., an outer diameter) of the body 1451.

The plunger assembly 1530 (FIG. 15B) is slidably disposed within the cannula assembly 1510 (FIG. 15A) such that that the plunger 1450 extends through the cannula 1440, the cannula hub 1515, and the plug 1516. The tip 1452 of the plunger 1450 has an outer dimension (e.g., outer diameter) that is larger than an inner dimension (e.g., inner diameter) of an opening extending through the plug 1516. In this manner, the plunger 1450 may move freely within the duct 1520 through the cannula 1440, the cannula hub 1515, and the plug 1516 until the tip 1452 of the plunger 1450 reaches the plug 1516, at which point the plug 1516 engages with the tip 1452 to stop the plunger 1450 from further movement. In an embodiment, a maximum outer dimension of the tip 1452 is about 0.38 mm (e.g., about 0.015 inch), a minimum inner diameter of the cannula 1440 is about 0.43 mm (e.g., about 0.017 inch), and a maximum inner diameter of the plug 1516 is about 0.30 mm (e.g., about 0.012 inch). In an embodiment, the plug 1516 has a material property such that the tip 1452 will deform the plug 1516 because it is slightly larger than the inner diameter of the plug 1516, and the plug 1516 will retain sufficient strength to keep the tip 1452 from continuing through and exiting the plug 1516.

Referring to FIG. 15E, a partially-assembled rail assembly 1540 includes, in an embodiment, the rail 1420, the extension spring 1430, the cannula assembly 1510, and the plunger assembly 1530. The extension spring 1430 includes an end, which, when the rail assembly 1540 is fully assembled, will be passed into the opening 1456 of the first interlock 1455 to couple the extension spring 1430 to the first interlock 1455. In this manner, movement of the first interlock 1455 will cause movement of the extension spring 1430 and movement of the extension spring 1430 will cause movement of the first interlock 1455.

The rail 1420 may have a distal end 1421 shaped to mate with a surface of the stand 1480 (or the housing when the stand 1480 is omitted) when the needle assembly 1400 is fully assembled. The rail 1420 defines lengthwise slits 1422 to allow at least the first interlock 1455 and the port 1441 to protrude from the rail 1420 as the respective plunger assembly 1530 and cannula assembly 1510 move within the rail 1420. The slits 1422 may be on opposite sides of the rail 1420, approximately 180 degrees apart from each other with respect to a lengthwise central axis of the rail 1420, or the slits 1422 may be defined at a different angle than 180 degrees. The rail 1420 may be open at a proximal end 1423 (as shown in the embodiment illustrated in FIG. 15C) to allow for ease of assembly of the components of rail assembly 1540. The rail assembly 1540 is assembled with the bar 1460 and other components to obtain the needle assembly 1400 of FIG. 14B.

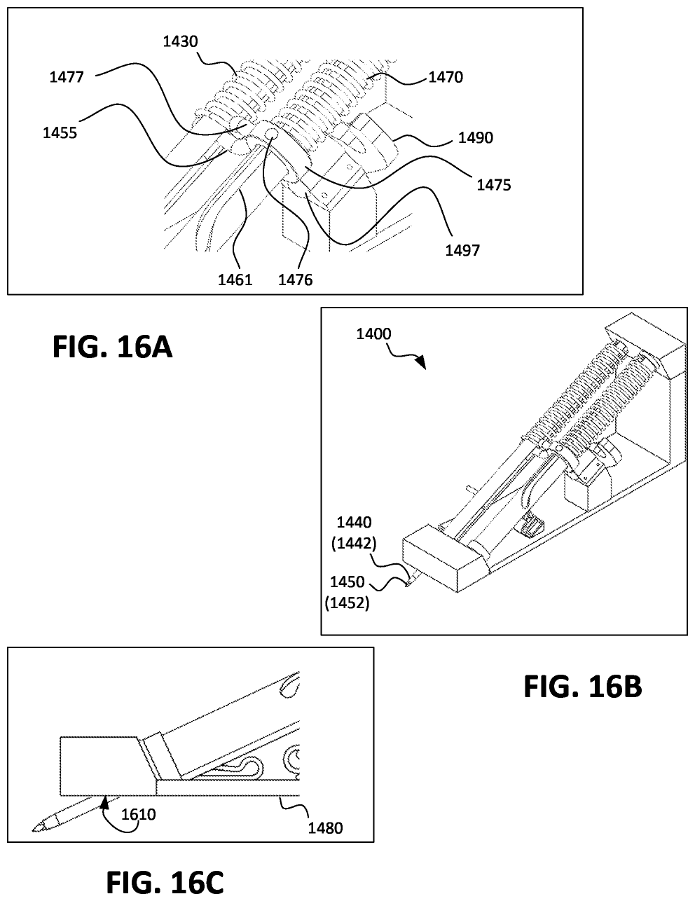

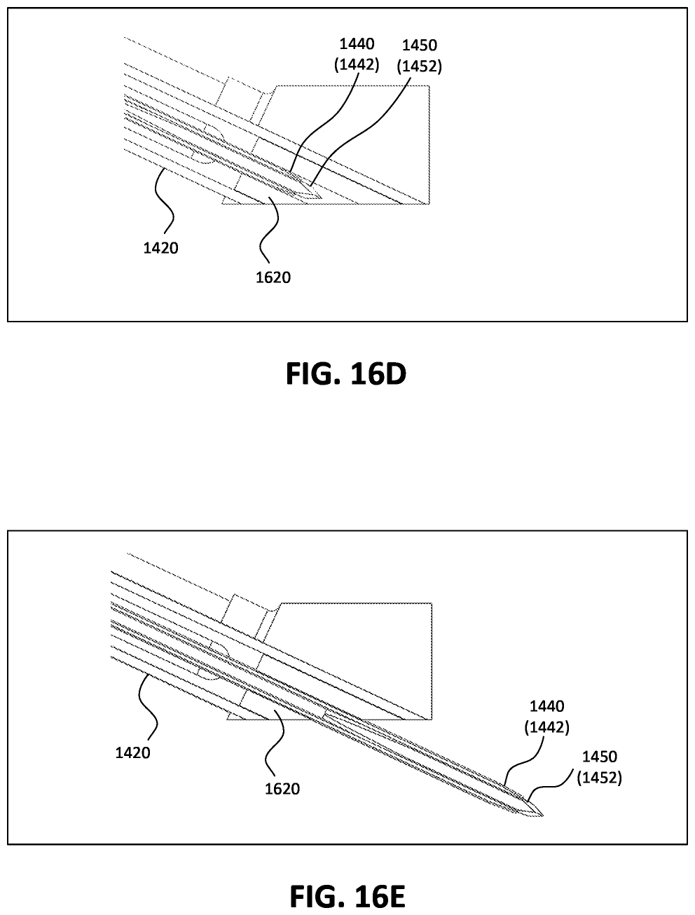

FIG. 16A-FIG. 16C illustrate a first phase of operation of the needle assembly 1400 subsequent to release of the retainer 1490.

Referring to FIG. 16A, the retainer 1490 is shown in a released state, rotated around the axis pin 1497, which has allowed the bushing 1475 to be pushed by the compression spring 1470 such that the slot post 1476 travels along the slot 1461. The first interlock 1455 and the second interlock 1477 are coupled together in this phase; as the compression spring 1470 decompresses towards its unbiased (relaxed) state, the extension spring 1430 is extended (biased).

Referring to FIG. 16B, as the compression spring 1470 decompresses and forces the extension spring 1430 to extend by way of the coupling of the first interlock 1455 and the second interlock 1477, the cannula 1440 and the plunger 1450 are forced out of the patch pump and through the skin of the subject. The tip 1452 of the plunger 1450 extends beyond the end 1442 of the cannula 1440 to create a channel through the layers of the skin to allow the cannula 1440 to be positioned in a configuration suitable for providing treatment.

Referring to FIG. 16C, the cannula 1440 and the plunger 1450 extend out of the patch pump through an opening 1610 defined in the stand 1480. In an embodiment, the stand 1480 is formed integrally with a housing (not shown) of the patch pump, and thus the opening 1610 is defined in the housing. In an embodiment, the stand 1480 is affixed to a housing (not shown) of the patch pump, and thus the housing also defines an opening, aligned with the opening 1610 in a manner to allow the cannula 1440 and the plunger 1450 to pass. Additionally, the patch pump may be disposed on an adhesive patch (not shown) for adhering the patch pump onto skin of the subject; in such embodiments, the adhesive patch will also define an opening aligned with the opening 1610 and/or the opening in the housing in a manner to allow the cannula 1440 and the plunger 1450 to pass. One or more of the openings allowing the cannula 1440 and the plunger 1450 to pass and therefore pierce the skin may be covered by a seal which the plunger 1450 pierces upon extension outside of the patch pump. In an embodiment, a seal is positioned over the opening 1610 or other opening at manufacture to maintain sterility of the patch pump until use.

FIG. 16D and FIG. 16E illustrate a comparison of an initial state (FIG. 16D) of the needle assembly of FIG. 14A and FIG. 14B versus a state of the needle assembly at or near an end of the first phase of operation (FIG. 16E) when both the cannula 1440 and the plunger 1450 have been extended to enter tissue of the subject.

Referring to FIG. 16D, in an initial state, the cannula 1440 and the plunger 1450 are maintained within the patch pump and within the rail 1420. In the embodiment illustrated, both the cannula 1440 and the plunger 1450 are maintained in a desired alignment with respect to the rail by a guide 1620 disposed within the rail 1420. In the embodiment illustrated, the tip 1452 of the plunger 1450 extends beyond the end 1442 of the cannula 1440 in the initial state; in other embodiments, the tip 1452 does not extend beyond the end 1442 in the initial state. In an embodiment, the tip 1452 extends about 0.8 mm (e.g., about 0.03 inch) beyond the end 1442 in the initial state. In an embodiment, the tip 1452 is flush with or recessed from the end 1442 in the initial state. In an embodiment, the tip 1452 extends from the end 1442 by less than about 0.25 mm (e.g., less than about 0.01 inch), or between about 0.25 mm to about 2.5 mm (e.g., between about 0.01 inch to about 0.10 inch).

Referring to FIG. 16E, at or near the end of the first phase of operation, the tip 1452 of the plunger 1450 is extended beyond a lower surface of the patch pump to pierce the skin and form a channel in tissue. The cannula 1440 is also extended, to advance into the channel formed by the tip 1452 of the plunger 1450.

FIG. 17A-FIG. 17C illustrate a second phase of operation of the needle assembly 1400 subsequent to release of the retainer 1490 and subsequent to the plunger 1450 and the cannula 1440 extending outside of the patch pump.

Referring to FIG. 17A, the first interlock 1455 and the second interlock 1477 remain coupled as the slot post 1476 of the bushing 1475 travels along the straight portion 1462 of the slot 1461 in the bar 1460.

Referring to FIG. 17B, as the slot post 1476 enters the curved portion 1463 of the slot 1461, the first interlock 1455 and the second interlock 1477 begin to decouple.

Referring to FIG. 17C, as the slot post 1476 follows the curvature of the curved portion 1463 of the slot 1461, the first interlock 1455 and the second interlock 1477 fully decouple, allowing the extension spring 1430 to return to its relaxed (not extended) state.

Because the end of the extension spring 1430 is passed within the opening 1456 of the first interlock 1455 and the first interlock 1455 is coupled to the plunger hub 1535 as described above, movement of the extension spring 1430 causes movement of the first interlock 1455 and the plunger hub 1535. Accordingly, when the extension spring returns towards its relaxed state (not extended), the plunger 1450 (which is coupled to the plunger hub 1535) is retracted into the cannula 1440, leaving the cannula 1440 positioned in or through the skin. The compression spring 1470 continues to decompress until it is stopped, such as by the slot post 1476 reaching the end of the curved portion 1463 of the slot 1461 of the bar 1460.

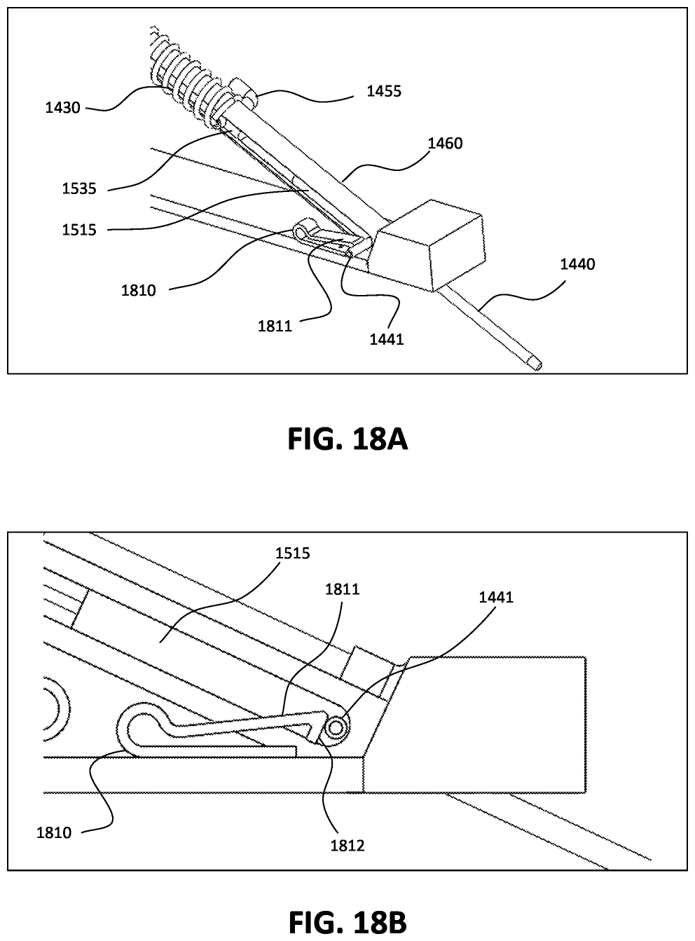

FIG. 18A and FIG. 18B illustrate a third phase of operation of the needle assembly 1400, subsequent to release of the retainer 1490 and subsequent to decoupling of the first interlock 1455 and the second interlock 1477. The cannula 1440 continues its motion through inertial forces. At the end of its inertial travel, the cannula 1440 could, if not hindered, be pushed back into the patch pump by tension in the tissue of the subject or by movement. A clip 1810 hinders such reversal. In the embodiment illustrated, the port 1441 runs along a side of the clip 1810 as the cannula 1440 extends out of the patch pump, and the port 1441 compresses an end 1811 of the clip 1810 as the port 1441 reaches the end 1811 and passes beyond the clip 1810. After the port 1441 passes the clip 1810, the end 1811 returns to its uncompressed state, and the end 1811 presents a face 1812 to the port 1441, hindering the port 1441 and thus the cannula 1440 from retracting into the patch pump. In an embodiment, rather than the port 1441 moving along the clip 1810 and being hindered by the face 1812 of the clip 1810, another protuberance coupled to the cannula assembly 1510 performs this function.

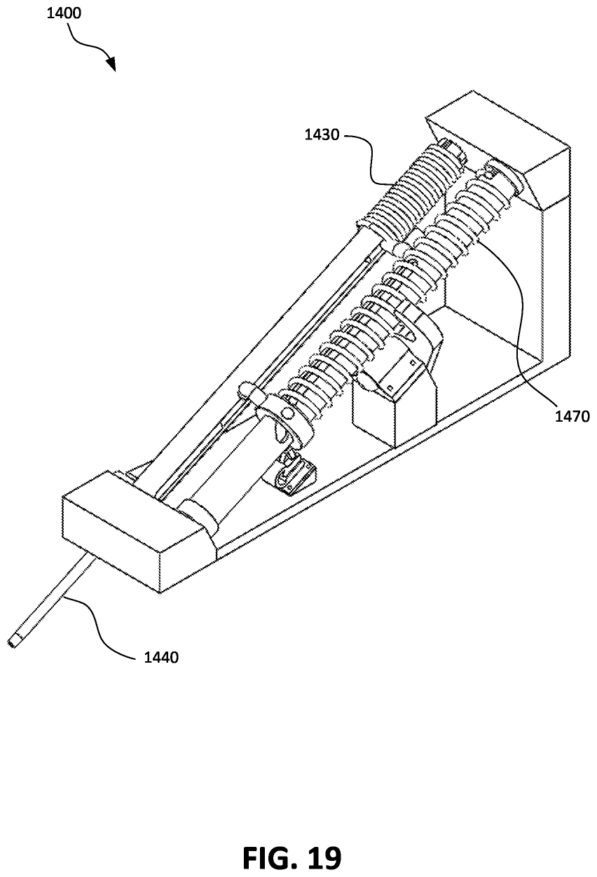

FIG. 19 illustrates an embodiment of the needle assembly 1400 in a final, relaxed state in which the compression spring 1470 is decompressed (is unbiased), the extension spring 1430 is not extended (is unbiased), and the cannula 1440 is positioned in tissue. Once the cannula 1440 is in place, a preparation can be delivered through the port 1441 and the cannula 1440. In this state, the tip 1452 of the plunger 1450 is retracted into the cannula 1440 past the guide 1620, and the plunger 1450 is held within the cannula hub 1515.

FIG. 20A-FIG. 20D illustrate an embodiment of using a pinch valve to block flow of a preparation from a reservoir until the cannula 1440 is in position.

Referring to FIG. 20A, a pinch valve 2020 pinches closed a pinch tube 2010. As the slot post 1476 of the bushing 1475 approaches the end of its travel within the slot 1461, the bushing 1475 encounters and opens the pinch valve 2020. The force exerted by the compression spring 1470 maintains the pinch valve 2020 in the open position. Other types of pinch valves may be used instead of pinch valve 2020. For example, a nitinol wire could be used, where the wire is in a closed (e.g., pinching) configuration until stimulated by an electrical current at which point the wire transforms to an open configuration (e.g., non-pinching), for electronic valve control.

Referring to FIG. 20B and FIG. 20C, the pinch tube 2010 fluidically couples together a flow tube 2030 and a connecting tube 2040. The flow tube 2030 is also fluidically coupled to at least one reservoir (not shown) that is internal to the patch pump, or to a feed port (not shown) that allows an external reservoir to be fluidically connected to the patch pump. The connecting tube 2040 is fluidically coupled to the port 1441, such as by a fluidic adapter 2015.

Referring to FIG. 20D, an example is shown of routing of the connecting tube 2040 from the port 1441 and under the rail 1420 and the bar 1460 to the pinch valve 2020. Such a routing path with a curvature can provide for strain relief to the connecting tube 2040. Hold-down components mounted over or around the connecting tube 2040 (not shown) may be implemented to provide additional strain relief. When the pinch valve 2020 is opened, a preparation can flow from a reservoir through a flowpath 2041 including the flow tube 2030, the pinch tube 2010 (not visible), the connecting tube 2040, the port 1441, and the cannula 1440.

In an embodiment, the pinch valve 2020 is a silicon tubing. In an embodiment, the flow tube 2030 and/or the connecting tube 2040 include polyurethane and/or polyvinyl chloride (PVC).

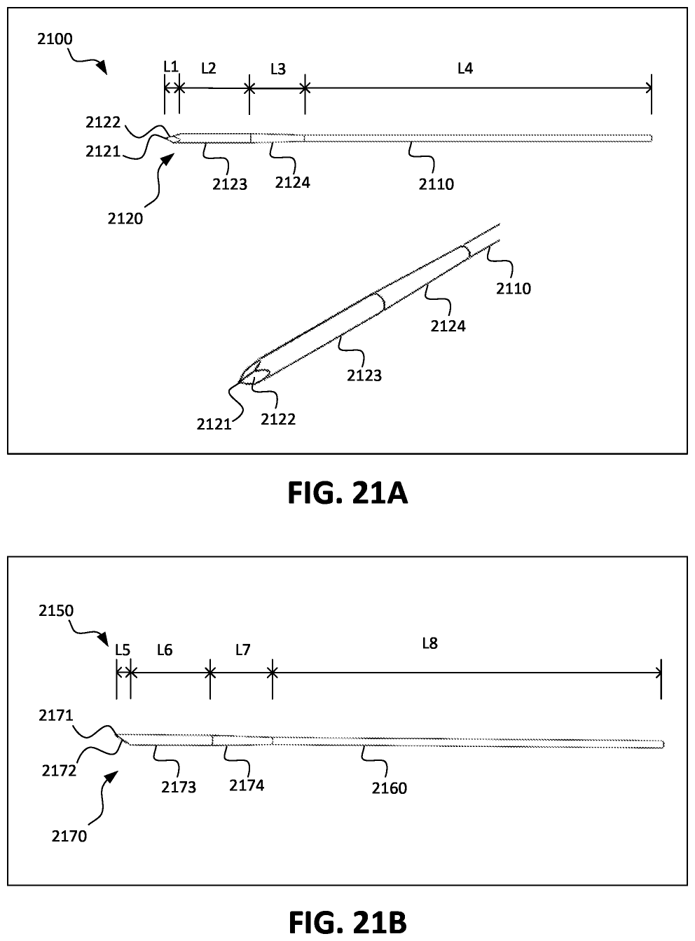

FIG. 21A and FIG. 21B illustrate examples of designs for a plunger (e.g., embodiments of the plunger 1450).

Referring to FIG. 21A, a plunger 2100 includes a shaft 2110 and a tip 2120. The tip 2120 includes a point 2121, one or more facets 2122, a segment 2123 including the facets 2122, and a joint 2124. The facets 2122 define a shape in which the point 2121 may be centered or offset. In an embodiment, a single facet defines a perimeter of the shape. In an embodiment, multiple facets define a perimeter of the shape. In an embodiment, the shape is similar to a trocar shape.

The point 2121 is sufficiently sharp to pierce through dermal layers. An increase in a length L1 of the facets 2122 may increase a sharpness of the point 2121. A hard coating (not shown) may fully or partially cover the tip 2120 to increase a hardness and/or sharpness of the point 2121. An intersection of different ones of the facets 2122 may be smooth or sharp. In an embodiment, the intersection between two of the facets 2122 is a sharp ridge which may serve to assist the point 2121 in piercing dermal layers.

The segment 2123 can have a substantially consistent cross-sectional shape and dimensions (e.g., area or diameter) along a length L2, although cross-sectional shape and dimensions may vary along the length L2.

In an embodiment, the joint 2124 transitions from matching the cross-sectional shape and dimensions (e.g., area or diameter) of the segment 2123 to matching a cross-sectional shape and dimensions (e.g., area or diameter) of the shaft 2110. The joint 2124 can have a substantially consistent cross-sectional shape along a length L3, although cross-sectional shape may vary along the length L3.

Dimensions of the shaft 2110 are sized to allow the shaft 2110 to move smoothly through the cannula 1440, the cannula hub 1515, and the plug 1516 of the cannula hub 1515. Slightly larger dimensions of the segment 2123 (or a portion of the segment 2123) are sized to allow the segment 2123 to move smoothly through the cannula 1440 and to be stopped within the plug 1516. In this manner, the segment 2123 is prevented from further movement by the plug 1516, and the segment 2123 together with the plug 1516 minimizes or prevents the preparation and/or biological matter passing from the cannula 1440 through the cannula hub 1515.

The length L1, the length L2, the length L3, and a length L4 of the shaft 2110 can be designed to accommodate a structure of an associated needle assembly. Cross-sectional shape and dimensions (e.g., area or diameter) of the facets 2122, the segment 2123, the joint 2124, and the shaft 2110 can be designed to accommodate a structure of an associated needle assembly.

In an embodiment, a maximum outer dimension (e.g., diameter) of the segment 2123 is from about 0.25 mm (e.g., about 0.010 inch) to about 0.40 mm (e.g., about 0.015 inch). In an embodiment, a minimum inner dimension (e.g., diameter of a lumen) of a cannula of an associated needle assembly is about 0.43 mm (e.g., about 0.017 inch). In an embodiment, a cannula of an associated needle assembly is a 24-gauge catheter and the segment 2123 is designed to slidably move within the catheter.

In an embodiment, one or more of the facets 2122, the segment 2123, the joint 2124, or the shaft 2110 may be separate components joined together to form the plunger 2100. In an embodiment, the facets 2122, the segment 2123, the joint 2124, and the shaft 2110 are integrally formed from a single length of material.

Referring to FIG. 21B, a plunger 2150 includes a shaft 2160 and a tip 2170. The tip 2170 includes a point 2171, a facet 2172, a segment 2173, and a joint 2174. The point 2171 is sufficiently sharp to pierce through dermal layers. An increase in a length L5 of the facet 2172 may increase a sharpness of the point 2171. A hard coating (not shown) may fully or partially cover the tip 2170 to increase a hardness and/or sharpness of the point 2171.

The segment 2173 can have a substantially consistent cross-sectional shape and dimensions (e.g., area or diameter) along a length L6, although cross-sectional shape and dimensions may vary along the length L6.

In an embodiment, the joint 2174 transitions from matching the cross-sectional shape and dimensions (e.g., area or diameter) of the segment 2173 to matching a cross-sectional shape and dimensions (e.g., area or diameter) of the shaft 2160. The joint 2174 can have a substantially consistent cross-sectional shape along a length L7, although cross-sectional shape may vary along the length L7.

Dimensions of the shaft 2160 are sized to allow the shaft 2160 to move smoothly through the cannula 1440, the cannula hub 1515, and the plug 1516 of the cannula hub 1515. Slightly larger dimensions of the segment 2173 (or a portion of the segment 2173) are sized to allow the segment 2173 to move smoothly through the cannula 1440 and be stopped within the plug 1516. In this manner, the segment 2173 is prevented from further movement by the plug 1516, and the segment 2173 together with the plug 1516 minimizes or prevents the preparation and/or biological matter passing from the cannula 1440 through the cannula hub 1515.

The length L5, the length L6, the length L7, and a length L8 of the shaft 2160 can be designed to accommodate a structure of an associated needle assembly. Cross-sectional shape and dimensions (e.g., area or diameter) of the facet 2172, the segment 2173, the joint 2174, and the shaft 2160 can be designed to accommodate a structure of an associated needle assembly.

In an embodiment, a maximum outer dimension (e.g., diameter) of the segment 2173 is from about 0.25 mm (e.g., about 0.010 inch) to about 0.40 mm (e.g., about 0.015 inch). In an embodiment, a minimum inner dimension (e.g., diameter of a lumen) of a cannula of an associated needle assembly is about 0.43 mm (e.g., about 0.017 inch). In an embodiment, a cannula of an associated needle assembly is a 24-gauge catheter and the segment 2173 is designed to slidably move within the catheter.

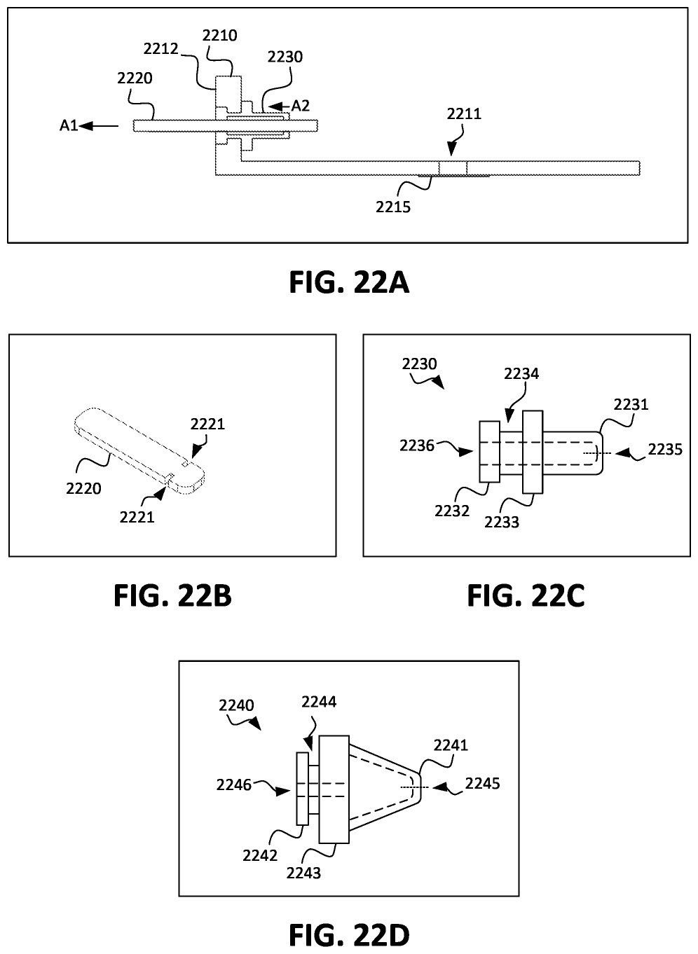

FIG. 22A-FIG. 22C illustrate an embodiment of a mechanism that can be used to activate a patch pump, such as to initiate balloon inflation and/or to initiate spring motion. A housing 2210 of a patch pump defines an opening 2211 which is covered by a seal 2215, through which a plunger (e.g., the plunger 142, 520, 620, 720, 1050, 1450, or 2450) and cannula (e.g., the cannula 144, 530, 630, 730, 1040, 1440, or 2440) can extend after actuation. The housing 2210, a tab 2220, and a stopper 2230 are assembled together. The tab 2220 defines at least one slot 2221. The stopper 2230 includes an end 2231 which protrudes into the interior of the patch pump when the stopper 2230 is assembled with the housing 2210. The stopper 2230 further includes flanges 2232, 2233 defining a gap 2234 therebetween. When assembled, a portion of a wall 2212 of the housing 2210 is positioned within the gap 2234 of the stopper 2230 such that the flanges 2232, 2233 abut the wall 2212 on opposite sides of the wall 2212. The stopper 2230 defines an opening 2235 in the end 2231, and further defines an aperture 2236 in fluidic communication with the opening 2235 and extending throughout the stopper 2230 from the flange 2232 to the end 2231. During assembly, the tab 2220 is pushed into the aperture 2236 and through the opening 2235 such that the slot(s) 2221 engage with the stopper 2230 within the opening 2235. The stopper 2230 is formed from one or more flexible materials to allow assembly of the stopper 2230 through the wall 2212 of the housing 2210, and to allow the opening 2235 to expand sufficiently for the tab 2220 to pass. The materials are resilient so that, in the absence of a deformation force, the stopper 2230 will substantially return to its original shape after deformation.

After assembly, when the tab 2220 is pulled in a direction away from the wall 2212 of the housing 2210 (e.g., in a direction indicated by arrow A1 in FIG. 22A), the engagement of the opening 2235 of the stopper 2230 with the slot(s) 2221 of the tab 2220 results in the end 2231 of the stopper 2230 deforming and moving in a direction towards the wall 2212 of the housing 2210 (e.g., in a direction indicated by arrow A2 in FIG. 22A). The stopper 2230 retains a seal around the tab 2220, as well as retaining a seal against the wall 2212 of the housing 2210. In this manner, an aseptic environment within the housing 2210 can be maintained when the tab 2220 is pulled. Pulling of the tab 2220 can pull on an attached component to activate the patch pump (e.g., activate inflation, and/or activate delivery of a plunger and cannula); for example, pull a switch or a lever or other triggering mechanism (e.g., the retainer 1490 or the pinch valve 2020).

Although the stopper 2230 and the tab 2220 are described with respect to pulling the tab 2220, in an alternative embodiment the tab 2220 is pushed to activate the patch pump (e.g., activate inflation, and/or activate delivery of a plunger and cannula). In such an embodiment, the end 2231 of the stopper 2230 is deformed to move with the tab 2220 in a direction away from the wall 2212 of the housing 2210 and further towards an interior of the patch pump (e.g., in a direction opposite that indicated by arrow A2). Such a movement can push the tab 2220 against another component to activate the patch pump (e.g., activate inflation and/or activate delivery of a plunger and cannula); for example, push a switch or a lever or other triggering mechanism (e.g., the retainer 1490 or the pinch valve 2020).

FIG. 22D illustrates an embodiment of a stopper 2240 that operates in a manner similar to the stopper 2230. The stopper 2240 includes an end 2241 defining an opening 2245, and flanges 2242, 2243 defining a gap 2244. The stopper 2240 further defines an aperture 2246 through which a tab (e.g., the tab 2220) is assembled such that one or more slots (e.g., the slot(s) 2221) of the tab engage within the opening 2245. The descriptions above of assembly and use of the stopper 2230 applies also to the stopper 2240.

A patch pump may include more than one stopper/tab mechanism (e.g., the stopper 2230 or 2240 and the tab 2220). A patch pump may include at least one pull tab and/or at least one push tab.

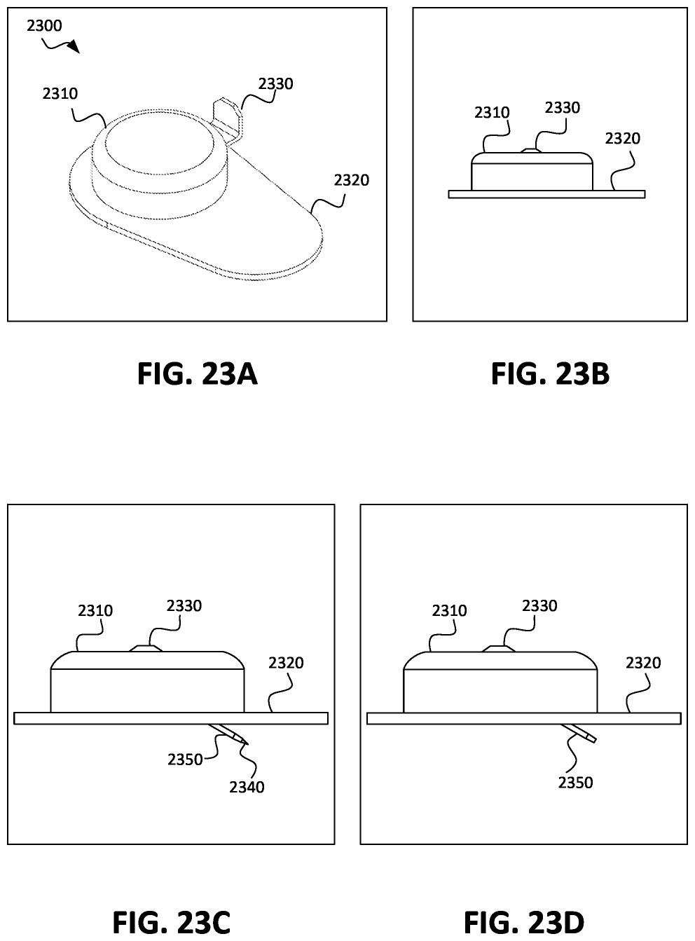

FIG. 23A and FIG. 23B illustrate exterior perspective and side views, respectively, of embodiments of a patch pump 2300. A housing 2310 is affixed to a patch 2320, such as an adhesive patch. In the embodiment illustrate in FIG. 23A and FIG. 23B, a footprint of the patch 2320 is significantly larger than a footprint of the housing 2310, perhaps a ratio of 2:1, which can provide a larger surface for handling the patch pump 2300, or a larger surface to apply against a skin surface, than would be provided by the footprint of the housing 2310. In other embodiments, a ratio of the footprint of the patch 2320 to the footprint of the housing 2310 would be less or greater, such as a ratio of 1:1, 1.5:1, 1:1.5, or 1:2. The patch pump 2300 includes a tab 2330 to pull or push, to activate the patch pump 2300.

FIG. 23C illustrates an embodiment of the patch pump 2300 of FIG. 23B during an intermediary phase of operation after the patch pump 2300 has been activated. In this phase, a plunger 2340 and a cannula 2350 extend beyond a lower surface of the patch 2320. The plunger 2340 extends beyond an end of the cannula 2350 to form a channel in tissue.

FIG. 23D illustrates an embodiment of the patch pump 2300 of FIG. 23B at a final state, in a configuration suitable for providing treatment. In this state, the plunger 2340 has been retracted within the patch pump 2300, and the cannula 2350 remains in the tissue to provide treatment.

FIG. 24A-FIG. 27B illustrate another embodiment (with variations) of a needle assembly of a patch pump. In this embodiment, the needle assembly is dual-spring and single-axis, with a compression spring that is extended in its relaxed state and can be biased into a compressed state and an extension spring that is not extended in its relaxed state and can be biased into an extended state. These embodiments are similar to needle assembly 1400 illustrated in FIG. 14A and FIG. 14B in many respects, with one difference being that the compression and extension springs in FIG. 24A-FIG. 27B move concentrically rather than in parallel as is the case for needle assembly 1400.

Initially, the compression spring is biased into a state of full or partial compression, and the extension spring is unbiased or substantially unbiased (e.g., in its relaxed state). The extension spring is coupled to a plunger of a needle assembly disposed in the patch pump. The compression spring is coupled to the extension spring. Upon the occurrence of an event, the compression spring is released from bias and extends towards its unbiased state, which biases the extension spring into an extended state and causes the plunger and a cannula to move. Movement of the compression spring deploys the needle assembly into tissue of a body (e.g., a pointed tip of the plunger advances into the tissue and creates a channel in the tissue, and the cannula enters the channel to deliver a preparation). After deployment, the compression spring is uncoupled from the extension spring, the compression spring subsequently remains in its unbiased state (extended), and the extension spring returns to its unbiased state (not extended). As the extension spring returns to its unbiased state (extended), the extension spring pulls the plunger back into the cannula.

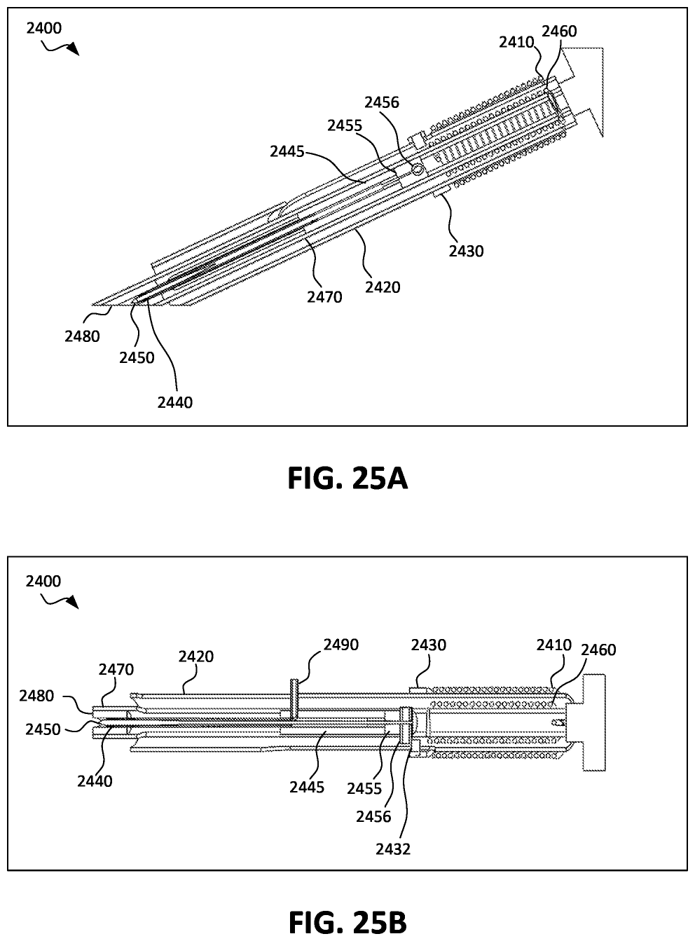

FIG. 24A-FIG. 24C illustrate general operation of the needle assembly 2400. FIG. 25A-FIG. 27B provide additional detail.

Referring now to FIG. 24A, an embodiment of a needle assembly 2400 is shown in perspective view as it might appear fully assembled and prior to activation. In this view, a compression spring 2410 is held in a compressed state by a bushing 2430, and both the compression spring 2410 and the bushing 2430 are movably disposed around a bar 2420. The bushing 2430 may be held in place until released by a release mechanism, such as described above with respect to FIG. 22A-FIG. 22D. When the release mechanism is released, the bushing 2430 no longer holds the compression spring 2410 in a compressed state, and the compression spring 2410 is allowed to expand to its relaxed state, pushing the bushing 2430 along the bar 2420.

Referring to FIG. 24B, movement of the compression spring 2410 along the bar 2420 causes a cannula 2440 and a plunger 2450 to extend from the needle assembly 2400 into tissue. The plunger 2450 enters the tissue first to create a tunnel in the tissue for the cannula 2440 to then enter.

In an embodiment, a tip of the plunger 2450 extends about 0.8 mm (e.g., about 0.03 inch) beyond an end of the cannula 2440 in an initial state (e.g., FIG. 24A). In an embodiment, the tip of the plunger 2450 is flush with or recessed from the end of the cannula 2440 in the initial state. In an embodiment, the tip of the plunger 2450 extends from the end of the cannula 2440 by less than about 0.25 mm (e.g., less than about 0.01 inch), or between about 0.25 mm to about 2.5 mm (e.g., between about 0.01 inch to about 0.10 inch). The plunger 2450 then extends into the tissue as determined by the constraints of the needle assembly 2400 and the patch pump in which it is disposed. For example, the plunger 2450 and/or the cannula 2440 may extend an amount approximately equal to the distance A in FIG. 11; other distances are within the scope of the present disclosure and the needle assembly 2400 may be designed to accommodate a length of the plunger 2450 suitable for an intended delivery mode (e.g., intramuscular, subcutaneous, or intravenous) or intended delivery site on the body (e.g., arm, leg, back, stomach, or buttock).

Referring to FIG. 24C, as the compression spring 2410 approaches or reaches full decompression, the plunger 2450 is retracted into the needle assembly 2400, and the cannula 2440 is left in the tissue to deliver a preparation from the patch pump (e.g., a preparation in one or more reservoirs within the patch pump, a preparation in a reservoir fluidically coupled to the patch pump, or a preparation in a syringe that is injected into tubing coupled to the cannula 2440).

FIG. 25A illustrates a side view of an embodiment of the needle assembly 2400 in lengthwise cross-section as it might appear fully assembled and prior to activation. The compression spring 2410 is held in a compressed state by the bushing 2430 along the bar 2420. A rail 2470 is positioned within the bar 2420. An extension spring 2460 is disposed over the rail 2470 in a relaxed, non-extended state. The cannula 2440 is attached to a cannula hub 2445. The plunger 2450 is positioned within the cannula 2440, and a plunger hub 2455 attached to the plunger 2450 is adjacent to the cannula hub 2445. The plunger 2450 and the cannula 2440 are held within the needle assembly 2400 until the compression spring 2410 is released. A guide 2480 is disposed within an end of the needle assembly 2400 to maintain the plunger 2450 and the cannula 2440 in an initial position until release, and to provide a trajectory path for the plunger 2450 and the cannula 2440 upon release.

FIG. 25B illustrates a top view of an embodiment of the needle assembly 2400 in lengthwise cross-section as it might appear fully assembled and prior to activation. In this view, a port 2490 is visible. The port 2490 is fluidically coupled to the cannula 2440.

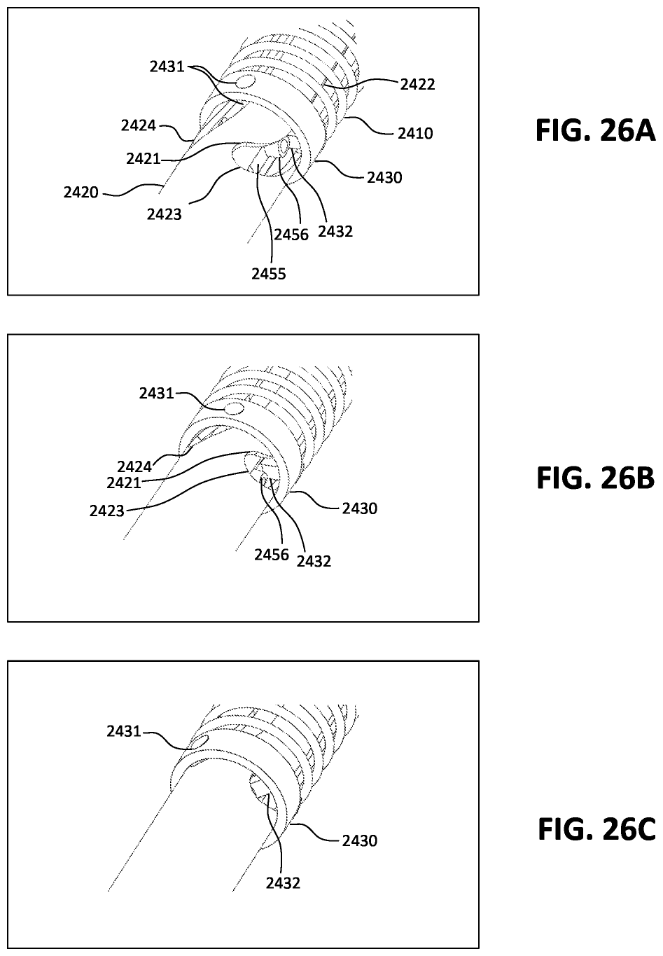

FIG. 26A-FIG. 26C illustrate engagement and disengagement features of the needle assembly 2400 (refer also to FIG. 25A and FIG. 25B). The bar 2420 includes a first slot 2421 and a second slot 2424. The first slot 2421 includes a straight portion 2422 and a curved portion 2423. A push post 2456 is coupled to the plunger hub 2455, which is coupled to the extension spring 2460. The bushing 2430 includes an alignment pin 2431 and a pushrod 2432. As the compression spring 2410 extends following its release from a compressed state (see, e.g., FIG. 24B and FIG. 24C), the pushrod 2432 pushes against the push post 2456 along the straight portion 2422 of first slot 2421 (FIG. 26A). Because the push post 2456 is coupled to the extension spring 2460, the movement of the push post 2456 along the first slot 2421 causes the extension spring 2460 to be pulled into an extended state. When the pushrod 2432 enters the curved portion 2423 of the first slot 2421 (FIG. 26B), the pushrod 2432 begins to disengage from the push post 2456 and eventually fully disengages (FIG. 26C) from the push post 2456. The disengagement of the pushrod 2432 and the push post 2456 allows the push post 2456 to move freely. Thus, because the extension spring 2460 is coupled to the push post 2456, the extension spring 2460 is allowed to return to its non-extended state. Further, because the push post 2456 is also coupled to the plunger hub 2455, when the extension spring 2460 returns to its non-extended state (see, e.g., the initial non-extended state of the extension spring 2460 in FIG. 25A) it pulls the plunger hub 2455 and thus the plunger 2450 out of the tissue and into the needle assembly 2400.

As the pushrod 2432 is pushed through the first slot 2421 by the compression spring 2410, the alignment pin 2431 travels along the second slot 2424. In an embodiment, the alignment pin 2431 and the second slot 2424 are omitted.

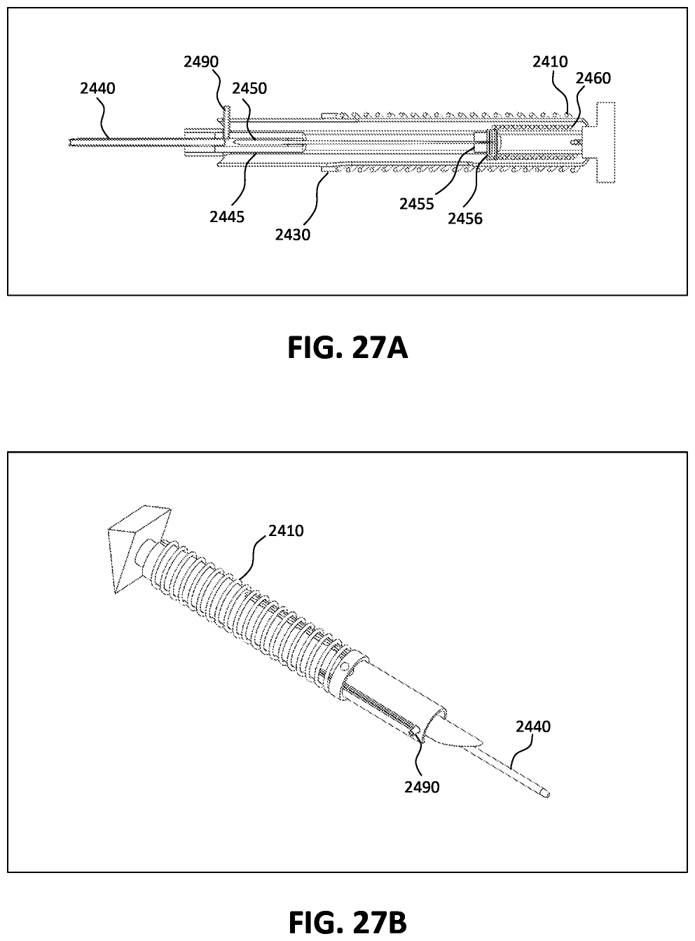

FIG. 27A illustrates a top view of an embodiment of the needle assembly 2400 in lengthwise cross-section (in a view angle similar to FIG. 25B) as it might appear after the plunger 2450 is retracted into the needle assembly 2400. The plunger 2450 is retracted fully within the cannula 2440. The port 2490 can be used to provide a preparation into the cannula hub 2445 and through the cannula 2440 into the tissue. A portion of the plunger 2450 is positioned and held within the cannula hub 2445 to block the preparation from flowing through the cannula hub 2445 and into the patch pump (see, e.g., the description of FIG. 21A and FIG. 21B).

FIG. 27B illustrates a perspective view of an embodiment of the needle assembly 2400 as it might appear after the plunger 2450 is retracted into the needle assembly 2400, at an angle where the port 2490 is visible.

Thus has been described several embodiments of a patch pump by way of example. Embodiments include, without limitation, the following:
A patch pump includes a needle assembly, a reservoir, a preparation, and an inflatable component. The needle assembly includes a cannula, and a plunger slidably disposed in the cannula. The plunger has a pointed tip and is structured to move within the cannula upon application of a force on the plunger such that the pointed tip of the plunger extends from the cannula to form a channel in a tissue of a subject. The reservoir is fluidically coupled to the needle assembly, and the preparation is contained in the reservoir. The inflatable component is structured to, upon inflation, press against the reservoir to force the preparation from the reservoir and into a flowpath between the reservoir and the cannula.
A needle assembly includes a compression spring, an extension spring, a cannula, a plunger slidably disposed in the cannula, and a port in fluidic communication with the cannula. The needle assembly is structured such that movement of the compression spring from a biased state to an unbiased state causes the plunger to move within the cannula and extend from the cannula to form a channel in tissue of a subject and causes the cannula to extend into the channel. The needle assembly may be used in a patch pump.
A method, includes, automatically by a patch pump when activated, forcing a plunger that is slidably disposed in a cannula to pierce a skin surface of a subject to create a channel through tissue of the subject, creating the channel through the tissue, forcing the cannula into the channel through the tissue, and releasing a pinch valve, thereby providing a flowpath from a reservoir to the cannula.
An embodiment may include any one of, or multiples of, or a combination of, the following features:
   The patch pump includes a spring initially in an unbiased state. The patch pump is structured such that the spring is biased into a compressed state by movement of the plunger within the cannula and then the spring automatically returns to its unbiased state, thereby applying a force on the plunger to reverse a direction of the plunger.
   The patch pump includes a spring initially in an unbiased state, the patch pump structured such that the spring is biased into an extended state by movement of the plunger within the cannula and then the spring automatically returns to its unbiased state, thereby applying a force on the plunger to reverse a direction of the plunger.
   The patch pump includes electronics structured to control a valve to controllably release the/a preparation from the/a flowpath into the cannula.
   The patch pump includes a normally-closed valve disposed in the cannula. The patch pump is structured to initially maintain the pointed tip of the plunger above the valve, allow the pointed tip to pass through and thus open the valve during movement of the plunger along a trajectory to extend the pointed tip from the cannula and form the channel, and return the pointed tip to a position above the valve subsequent to forming the channel, thus closing the valve.
   The patch pump includes electronics structured to receive a signal from an external device and responsively cause the/an inflatable component to inflate and thereby cause delivery of the/a preparation to the subject.
   The patch pump is structured such that movement of the plunger within the cannula causes the cannula to advance into the channel in the tissue formed by the plunger.
   In the/a needle assembly, the/a compression spring moves along a first trajectory and the/an extension spring moves along a second trajectory. The first trajectory and the second trajectory may form an angle. The angle may be in a range of about 15 degrees to about 75 degrees. Alternatively, the first trajectory and the second trajectory may be parallel to each other. The compression spring and the extension spring may be concentric such that the first trajectory and the second trajectory are approximately the same trajectory.
   The/a needle assembly includes a retainer, and the/a compression spring is released to move along the/a first trajectory upon release of the retainer.
   A movement of the/a compression spring from a biased state to an unbiased state releases a pinch valve to allow fluid to flow from a reservoir into the cannula.
   The patch pump, automatically when activated, mixes two reactants, thereby generating a gas.
   The patch pump, automatically when activated, inflates an inflatable component using a generated gas.
   The patch pump, automatically when activated, uses an inflatable component to apply pressure to the/a reservoir, thereby forcing fluid from the reservoir into the/a flowpath.
   The patch pump, automatically when activated, uses an inflatable component to apply pressure to the plunger to force the plunger to pierce the/a skin surface.
   The plunger may be hollow or solid.
   The patch pump includes multiple needle assemblies, each with its own reservoir or reservoirs.
   The patch pump includes multiple needle assemblies associated with a shared reservoir or with multiple shared reservoirs, such that delivery of fluid from the reservoir(s) may be faster than delivery through a single needle assembly.
   The patch pump includes one needle assemble including multiple cannulas and multiple plungers, each plunger disposed in a separate cannula.
   The patch pump includes one needle assemble including one cannula and multiple plungers, the plungers disposed in the cannula.
   A kit includes the patch pump and a reservoir to be fluidically coupled to the patch pump. The reservoir may be pre-filled with a preparation, or the kit may contain a mechanism such as a syringe to fill the reservoir with a preparation, where the preparation may be separately provided or provided in the kit.
   A kit includes the patch pump. A reservoir in the patch pump may be pre-filled with a preparation, or the kit may contain a mechanism such as a syringe to fill the reservoir with a preparation, where the preparation may be separately provided or provided in the kit.
   The plunger and cannula may be sized and oriented within the patch pump to deliver medication cutaneously, subcutaneously, intramuscularly, or intravenously.
   The patch pump, or one or more components of the patch pump, are disposable.
   One or more components of the patch pump are degradable.
   A patch pump may include one or more springs for forcing the plunger out of the patch pump or retracting the plunger into the patch pump, such as fly, torsion, helical, spiral, linear, progressive, dual rate, flat, disc, conical, volute, leaf, bar, Belleville, wave, constant force, or power springs. When multiple springs are used, similar or different types of springs may be employed.
   The/a reservoir is external to the patch pump. In such a configuration, dimensions of the patch pump may be, for example: a height of up to about 10 mm (e.g., about 0.39 inch); and an oval base with (short)×(long) axis lengths of up to about 10×20 mm (e.g., about 0.39×0.79 inch).
   The/a reservoir is internal to the patch pump. In such a configuration, dimensions of the patch pump may be, for example: a height of up to about 15.0 mm (e.g., about 0.59 inch); and an oval base with (short)×(long) axis lengths of up to about 20×40 mm (e.g., about 0.79×1.57 inch). For another example, dimensions of the patch pump may be a height of up to about 13.0 mm (e.g., about 0.51 inch) and a circular radius of about 38.0 mm (e.g., about 1.50 inches).
   The patch pump has a height up to about 50 mm (e.g., about 2 inches) and a width or length up to about 100 mm (e.g., about 4 inches).
   The/a reservoir contains a single dose of a medication.
   The/a reservoir contains multiple doses of a medication, and the patch pump is programmed to deliver doses at pre-programmed times or intervals.
   The/a reservoir contains multiple doses of a medication, and the patch pump is structured to meter the multiple doses out through the cannula continuously at a slow rate, over a time period in a range of about 5 minutes to about 7 days.
   The patch pump may be in any suitable size, shape, or configuration. For example, a size of the patch pump may be selected from a variety of available sizes based on a reservoir size to provide a therapeutic amount of a medication in a preparation on a per kg basis.

Thus has been described embodiments of a patch pump that provides for delivery of preparation through skin of a subject. Examples of use include:
A quantity of patch pumps is delivered to a region that has limited access to a healthcare facility, to enable inhabitants of the region to administer a vaccine to themselves or to others.
A quantity of patch pumps is delivered to a quarantined area to enable quarantined persons to administer a vaccine or other preparation to themselves and thereby avoid contact with caregivers for the administration.
A quantity of patch pumps is delivered to a facility or organization to facilitate mass vaccination of a population.
A care provider administers a preparation to a subject using a patch pump in a caregiver facility or at the subject's home.
A patch pump is adhered to a subject's arm to deliver insulin when low blood sugar of the subject is detected.
A patch pump detects a blood sugar level of a subject and triggers a delivery of insulin to the subject when the blood sugar level crosses a threshold.
A patch pump detects a blood oxygen level of a subject and triggers a delivery of microparticles containing oxygen gas when the blood oxygen level crosses a threshold.
A patch pump inserts a cannula into tissue of a subject; the cannula is coupled to a port exposed externally to the patch pump, and a preparation is injected into the exposed port and thus into the tissue of the subject.
A patch pump includes a first reservoir containing a first preparation, and a second reservoir containing a second preparation; the first preparation is delivered by the patch pump at a first time or first trigger event, and the second preparation is delivered by the patch pump at a second time or second trigger event.
A computing device (e.g., smartphone or smart watch or computer) initiates a trigger event by sending a signal to electronics in a patch pump to deliver preparation to a subject.
A patch pump delivers biotherapeutic agents requiring parenteral delivery, in place of frequent/daily injections.
A patch pump is selected for use to provide for tight titration, meaning low variation of an amount of medication to be administered.
A patch pump is selected to replace oral delivery of medication for a pediatric patient (e.g., less than 10 years old) who are unable to take drugs orally.
Patch pumps are carried as an item in a medical kit, such as for emergency, field, or military use to provide rescue medication.
A patch pump including a reservoir containing naloxone is affixed to skin of a patient and activated to reverse effects of opioids by the naloxone binding to sites that bind opioids. For example, during surgery a patient may be given opioids (anesthesia), and the patch pump is activated post-surgery to deliver the naloxone to the patient. The naloxone may be delivered in an initial bolus and optionally a subsequent slow delivery over several hours, such up to about 10 hours, because opioids have a long half-life so the effects of the opioids continue for a long time. The slow delivery may be a constant slow delivery, or an occasional release of a small amount of naloxone. The patch pump could stay affixed to the skin of the patient even after being released from the hospital, to continue to deliver the naloxone until the reservoir is empty.

Having provided several embodiments of the patch pump by way of example, a few definitions are now provided for the reader's reference.

Various abbreviations are used herein for standard units, such as deciliter (dl), milliliter (ml), microliter (μl), international unit (IU), cubic centimeter (cc), centimeter (cm), millimeter (mm), kilogram (kg), gram (gm), milligram (mg), microgram (μg), millimole (mM), degrees Celsius (° C.), millitorr (mTorr), hour (hr), or minute (min).

When used in the present disclosure, the terms “e.g.,” “such as”, “for example”, “for an example”, “for another example”, “examples of”, “by way of example”, and “etc.” indicate that a list of one or more non-limiting example(s) precedes or follows; it is to be understood that other examples not listed are also within the scope of the present disclosure.

As used herein, the singular terms “a,” “an,” and “the” may include plural referents unless the context clearly dictates otherwise. Reference to an object in the singular is not intended to mean “one and only one” unless explicitly so stated, but rather “one or more.”

The term “in an embodiment” or a variation thereof (e.g., “in another embodiment” or “in one embodiment”) refers herein to use in one or more embodiments, and in no case limits the scope of the present disclosure to only the embodiment as illustrated and/or described. Accordingly, a component illustrated and/or described herein with respect to an embodiment can be used in another embodiment (e.g., in another embodiment illustrated and described herein, or in another embodiment within the scope of the present disclosure and not illustrated and/or not described herein).

The term “component” refers herein to one item of a set of one or more items that together make up a device, formulation, preparation, or system under discussion. A component may be in a solid, powder, gel, plasma, fluid, gas, or other form. For example, a device may include multiple solid components which are assembled together to structure the device and may further include a liquid component that is disposed in the device. For another example, a preparation may include two or more powdered and/or fluid components which are mixed together to make the preparation.

The term “design” or a grammatical variation thereof (e.g., “designing” or “designed”) refers herein to characteristics intentionally incorporated based on, for example, estimates of tolerances (e.g., component tolerances and/or manufacturing tolerances) and estimates of environmental conditions expected to be encountered (e.g., temperature, humidity, external or internal ambient pressure, external or internal mechanical pressure, stress from external or internal mechanical pressure, age of product, or shelf life, or, if introduced into a body, physiology, body chemistry, biological composition of fluids or tissue, chemical composition of fluids or tissue, pH, species, diet, health, gender, age, ancestry, disease, or tissue damage); it is to be understood that actual tolerances and environmental conditions before and/or after delivery can affect characteristics so that different components, devices, preparations, or systems with a same design can have different actual values with respect to those characteristics. Design encompasses also variations or modifications before or after manufacture.

The term “manufacture” or a grammatical variation thereof (e.g., “manufacturing” or “manufactured”) as related to a component, device, preparation, or system refers herein to making or assembling the component, device, preparation, or system. Manufacture may be wholly or in part by hand and/or wholly or in part in an automated fashion.

The term “structured” or a grammatical variation thereof (e.g., “structure” or “structuring”) refers herein to a component, device, preparation, or system that is manufactured according to a concept or design or variations thereof or modifications thereto (whether such variations or modifications occur before, during, or after manufacture) whether or not such concept or design is captured in a writing.

The term “body” refers herein to an animalia body.

The term “subject” refers herein to a body into which an embodiment of the present disclosure is, or is intended to be, delivered. For example, with respect to humans, a subject may be a patient under treatment of a health care professional.

The term “fluid” refers herein to a liquid or gas, and encompasses moisture and humidity. The term “fluidic environment” refers herein to an environment in which one or more fluids are present.

The term “biological matter” refers herein to blood, tissue, fluid, enzymes, interstitial fluid, and other secretions of a body.

The term “preparation” refers herein to a medicinal preparation (e.g., including one component or a combination of components) intended for a therapeutic, diagnostic, or other biological purpose in any form. A preparation may be in a liquid form, a powder form, or a condensed or a consolidated form such as a tablet or microtablet. Each preparation can include one or more components, and a device or system can include one or more preparations. A component of a preparation can be, for example, a pharmacologically active agent, a DNA or SiRNA transcript, a cell, a cytotoxic agent, a vaccine or other prophylactic agent, a nutraceutical agent, a vasodilator, a vasoconstrictor, a delivery enhancing agent, a delay agent, an excipient, a diagnostic agent, or a substance for cosmetic enhancement.

A pharmacologically active agent can be, for example, an antibiotic, a nonsteroidal anti-inflammatory drug (NSAID), an angiogenesis inhibitor, a neuroprotective agent, a chemotherapeutic agent, a peptide, a protein, an immunoglobulin (e.g., a TNF-alpha antibody), an interleukin in the IL-17 family of interleukins, an anti-eosinophil antibody, another antibody, a nanobody, a large molecule, a small molecule, or a hormone, or a biologically active variant or derivative of any of the foregoing.

A cell can be, for example, a stem cell, a red blood cell, a white blood cell, a neuron, or other viable cell. Cells can be produced by or from living organisms or contain components of living organisms. A cell can be allogeneic or autologous.

A vaccine can be, for example, against an influenza, a coronavirus, meningitis, human papillomavirus (HPV), or chicken pox. A vaccine can correspond to an attenuated virus.

A nutraceutical agent can be, for example, vitamin A, thiamin, niacin, riboflavin, vitamin B-6, vitamin B-12, another B-vitamin, vitamin C (ascorbic acid), vitamin D, vitamin E, folic acid, phosphorous, iron, calcium, or magnesium.

A vasodilator can be, for example, I-arginine, sildenafil, a nitrate (e.g., nitroglycerin), or epinephrine.

A vasoconstrictor can be, for example, a stimulant, an amphetamine, an antihistamine, epinephrine, or cocaine.

A delivery enhancement agent can be, for example, a permeation enhancer, an enzyme blocker, a peptide that permeates through mucosa, an antiviral drug such as a protease inhibitor, a disintegrant, a superdisintegrant, a pH modifier, a surfactant, a bile salt, a fatty acid, a chelating agent, or a chitosan. A delivery enhancing agent can, for example, serve as a delivery medium for delivery of a component of a preparation, or serve to improve absorption of a component of a preparation into the body. A delivery enhancing agent can prime an epithelium of the intestine (e.g., fluidize an outer layer of cells) to improve absorption and/or bioavailability of one or more other components included in the delivery device.

A delay agent can be, for example, poly(lactic acid) (PLA), poly(glycolic acid) (PGA), polyethylene glycol (PEG), poly(ethylene oxide) (PEO), poly (I-lactic acid) (PLLA), poly(D-lactic acid) (PDLA), another polymer, or a hydrogel. A delay agent can be included with (e.g., mixed with, or providing a structure around) one or more other component(s) in a preparation to slow a release rate of the other component(s) from the preparation.

An excipient can be, for example, a binder, a disintegrant, a superdisintegrant, a buffering agent, an anti-oxidant, or a preservative. Excipients can provide a medium for a component of a preparation (e.g., for assisting in manufacture), or to preserve integrity of a component of a preparation (e.g., during manufacture, during storage, or after ingestion prior to dispersion within the body).

A diagnostic agent can be, for example, a sensing agent, a contrast agent, a radionuclide, a fluorescent substance, a luminescent substance, a radiopaque substance, or a magnetic substance.

The terms “substantially” and “about” are used herein to describe and account for small variations. For example, when used in conjunction with a numerical value, the terms can refer to a variation in the value of less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values may sometimes be presented herein in a range format. As used herein, a range of numbers includes any number within the range, or any sub-range if the minimum and maximum numbers in the sub-range fall within the range. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. Thus, for example, “<9” can refer to any number less than nine, or any sub-range of numbers where the minimum of the sub-range is greater than or equal to zero and the maximum of the sub-range is less than nine. Ratios may also be presented herein in a range format. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The foregoing description of various embodiments has been presented for purposes of illustration and description. It is not intended to limit the invention to the precise forms disclosed. Many modifications, variations and refinements will be apparent to practitioners skilled in the art. For example, embodiments of the device can be sized and otherwise adapted for various pediatric and neonatal applications as well as various veterinary applications. Also, those skilled in the art will recognize, or be able to ascertain using no more than routine experimentation, numerous equivalents to the specific devices and methods described herein. Such equivalents are considered to be within the scope of the present invention and are covered by the appended claims below.

Accordingly, while the present disclosure has been described and illustrated with reference to specific embodiments thereof, these descriptions and illustrations are not limiting. It can be clearly understood that various changes can be made, and equivalent components can be substituted within the embodiments, without departing from the true spirit and scope of the invention as defined by the appended claims. Also, components, characteristics, or acts from one embodiment can be readily recombined or substituted with one or more components, characteristics or acts from other embodiments to form numerous additional embodiments within the scope of the invention. Moreover, components that are shown or described as being combined with other components, can, in various embodiments, exist as standalone components. Further, for any positive recitation of a component, characteristic, constituent, feature, step or the like, embodiments of the invention specifically contemplate the exclusion of that component, value, characteristic, constituent, feature, step or the like. The illustrations may not necessarily be drawn to scale. There can be distinctions between the artistic renditions in the present disclosure and the actual apparatus, due to variables in manufacturing processes and such. There can be other embodiments of the present disclosure which are not specifically illustrated. The specification and drawings are to be regarded as illustrative rather than restrictive. Modifications can be made to adapt a particular situation, material, composition of matter, method, or process to the objective, spirit and scope of the present disclosure. All such modifications are intended to be within the scope of the claims appended hereto. While the methods disclosed herein have been described with reference to particular operations performed in a particular order, it can be understood that these operations can be combined, sub-divided, or re-ordered to form an equivalent method without departing from the teachings of the present disclosure. Therefore, unless specifically indicated herein, the order and grouping of the operations are not limitations.

1. A patch pump, comprising:
a needle assembly comprising a cannula and further comprising a plunger slidably disposed in the cannula, the plunger having a pointed tip, the plunger structured to move within the cannula upon application of a force on the plunger such that the pointed tip of the plunger extends from the cannula to form a channel in a tissue of a subject;
a reservoir fluidically coupled to the needle assembly;
a preparation contained in the reservoir; and
an inflatable component structured to, upon inflation, press against the reservoir to force the preparation from the reservoir and into a flowpath between the reservoir and the cannula.

2. The patch pump of claim 1, further comprising a spring, and wherein the patch pump is structured such that (i) the spring is biased into a compressed state by movement of the plunger within the cannula, and (ii) the spring automatically returns from the compressed state to an unbiased state, thereby applying a force on the plunger to reverse a direction of the plunger.

3. The patch pump of claim 1, further comprising a spring, and wherein the patch pump is structured such that (i) the spring is biased into an extended state by movement of the plunger within the cannula, and (ii) the spring automatically returns from the extended state to an unbiased state, thereby applying a force on the plunger to reverse a direction of the plunger.

4. The patch pump of claim 1, further comprising electronics structured to control a valve to controllably release the preparation from the flowpath into the cannula.

5. The patch pump of claim 1, further comprising a normally-closed valve disposed in the cannula, the patch pump structured to initially maintain the pointed tip of the plunger above the valve, allow the pointed tip to pass through and thus open the valve during movement of the plunger along a trajectory to extend the pointed tip from the cannula and form the channel, and return the pointed tip to a position above the valve subsequent to forming the channel, thus closing the valve.

6. The patch pump of claim 1, further comprising electronics structured to receive a signal from an external device and responsively cause the inflatable component to inflate and thereby cause delivery of the preparation to the subject.

7. The patch pump of claim 1, wherein the patch pump is structured such that movement of the plunger within the cannula causes the cannula to advance into the channel in the tissue formed by the plunger.

8. A needle assembly, comprising:
a compression spring;
an extension spring;
a cannula;
a plunger slidably disposed in the cannula; and
a port in fluidic communication with the cannula, wherein the needle assembly is structured such that movement of the compression spring from a biased state to an unbiased state causes the plunger to move within the cannula and extend from the cannula to form a channel in tissue and the cannula to extend into the channel.

9. The needle assembly of claim 8, wherein the compression spring moves along a first trajectory and the extension spring moves along a second trajectory.

10. The needle assembly of claim 9, wherein the first trajectory and the second trajectory form an angle.

11. The needle assembly of claim 10, wherein the angle is in a range of 15 degrees to 60 degrees.

12. The needle assembly of claim 9, wherein the first trajectory and the second trajectory are parallel to each other.

13. The needle assembly of claim 9, the compression spring and the extension spring are concentric such that the first trajectory and the second trajectory are approximately the same trajectory.

14. The needle assembly of claim 9, further comprising a retainer, wherein the compression spring is released to move along the first trajectory upon release of the retainer.

15. The needle assembly of claim 8, further comprising a reservoir and a pinch valve, wherein the movement of the compression spring from a biased state to an unbiased state releases the pinch valve to allow fluid to flow from the reservoir into the cannula.

16. A method, comprising, automatically by a patch pump when activated:
forcing a plunger that is slidably disposed in a cannula to pierce a skin surface of a subject to create a channel through tissue of the subject;
creating the channel through the tissue;
forcing the cannula into the channel through the tissue; and
releasing a pinch valve, thereby providing a fluidic path from a reservoir to the cannula.

17. The method of claim 16, further comprising, automatically by the patch pump when activated, mixing two reactants, thereby generating a gas.

18. The method of claim 17, further comprising, automatically by the patch pump when activated, inflating an inflatable component using the generated gas.

19. The method of claim 18, further comprising, automatically by the patch pump when activated, using the inflatable component to apply pressure to the reservoir, thereby forcing fluid from the reservoir into the fluidic path.

20. The method of claim 18, further comprising, automatically by the patch pump when activated, using the inflatable component to apply pressure to the plunger to force the plunger to pierce the skin surface.
